(19) United States
(12) Patent Application Publication
(10) Pub. No.: US 2005/0118611 A1
(43) Pub. Date: Jun. 2, 2005

(54) Thioaptamers enable discovery of physiological pathways and new therapeutic strategies

(75) Inventors: David G. Gorenstein, Houston, TX (US); Bruce A. Luxon, Galveston, TX (US)

Correspondence Address: CHALKER FLORES, LLP, 12700 PARK CENTRAL, STE. 455, DALLAS, TX, 75251, US

(73) Assignee: Board Of Regents, The University of Texas System, Austin, TX (US)

(21) Appl. No.: 10/898,470

(22) Filed: Jul. 23, 2004

Related U.S. Application Data
(60) Provisional application No. 60/489,663, filed on Jul. 24, 2003.

(51) Int. Cl. C12Q 1/68; C07H 21/02; C12N 15/85

(52) U.S. Cl. 435/6; 435/455; 536/23.1

(57) ABSTRACT

A system and method for determining the effect of a target molecule within a physiological pathway is disclosed in which a thioaptamer is used to affect a detectable activity of the physiological pathway in a cell or cell fragment.

This application claims priority to Provisional Patent Application Ser. No. 60/489,663, filed Jul. 24, 2004. Without limiting the scope of the invention, its background is described in connection with oligonucleotide agents and with methods for the isolation and generation thereof.

The U.S. Government may own certain rights in this invention pursuant to the terms of the DARPA (9624-107 FP) and NIH (A127744).

### TECHNICAL FIELD OF THE INVENTION

The present invention relates in general to the field of thioaptamers, and more particularly, to the use of thioaptamers for drug discovery, evaluation and characterization of physiological pathways.

### BACKGROUND OF THE INVENTION

With the advent of the description of the human genome and the emergence of high throughput analytical techniques such as genomic microarrays and proteomics gels and chips, a vast wealth of data on the functions of biological systems is quickly emerging. A result of the genome project is that a major paradigm shift in biological and biomedical discovery will continue and expand over many future decades, if not centuries. The impact on human health and the management of disease is almost incalculable.

Central to this revolution in health science is the discovery of detail of the pathways through which physiological systems communicate. It is a central premise of the Human Genome Project that understanding the genes that underlie the biological pathways that operate and interconnect, biomedical researchers can learn how to target specific nodes (often proteins) in these pathways, in order to modulate their activity through the use of drugs. The power of this genetic knowledge is enormous and promises to enable the targeting of therapies for disease, aging and other physiological circumstances through molecular medicine.

Unfortunately, present technology fails to enable biomedical researchers to dissect exceedingly complex physiological pathways in sufficient detail to identify targets for therapy. Simple organisms lend themselves to the genetic engineering necessary to construct a sufficient number of genetic variants (hundreds to thousands) so that scientists can probe the putative pathways through which these organisms maintain homeostasis, respond to diseases, and sustain life. Such simple systems rarely shed much light on how more complex organisms operate, at a level sufficient to support drug discovery.

Typically, the systems studied have been either prokaryotic or exceedingly simple single cell eukaryotic organisms (e.g. yeast), that are insufficiently complex to aid human biomedical research in a practical way. The major barrier to such research on complex, multi-cellular systems has been the technological and ethical barriers to genetically engineering large numbers of groups containing known genetic mutations at specific loci. Oftentimes, when attempted in lower species such as mice, these “knockouts” are lethal and the effects of the knockout are so diverse as to be only moderately informative. The long-term value of such genetically engineered animal models is clearly limited, and obviously not applicable to humans.

Thus, there is a need in the art for systems and methods for detecting binding affinity for a target molecule and detectable read-out systems that permit dissection of the effects of molecules on particular biochemical and/or physiological pathways. Also needed are ways to identify and quantify in detail the mechanisms by which candidate molecules interact with the biochemistry and physiology of cells or cell fractions.

### SUMMARY OF THE INVENTION

The present invention is a system and method for monitoring biological interactions and dissecting biochemical and physiological pathways. The system and method includes contacting a thio-modified nucleotide aptamer or thioaptamer that specifically binds to a target molecule or portion thereof and detecting the physiological effects of the interactions between the thioaptamer and target molecule or portion thereof. Detection of the effects of thioaptamer binding may be detected at a variety of levels and using a variety of read-outs as disclosed herein. Generally, modulation of the functional attributes of bioactive targets is achieved by thioaptamer binding. Binding may, for example, interrupt protein DNA interactions such as those that occur between transcription factors and DNA in the process of gene activation. The ability to modulate effectively the effects of certain pluripotent transcription factors in vivo would provide a particularly valuable therapeutic tool.

The present invention includes a method of disrupting a target molecule within a physiological pathway that includes the steps of characterizing a detectable activity of the physiological pathway, identifying a thioaptamer that modifies the activity of the target molecule of the physiological pathway and determining the extent of the modification to the activity of the physiological pathway in the presence of the thioaptamer. The thioaptamer may include between one to about 67% of the phosphate links of the thioaptamer which are thio-modified on one or both strands. The thioaptamer may be single or double stranded, DNA, RNA or PNA and even an siRNA, such as an miRNA or an RNAi. The target molecule may be, e.g., a nucleic acid, a protein, a complex of proteins and a complex of one or more proteins and one or more nucleic acids. The detectable activity that is measured may be determined using a chemical assay and/or a biological assay or may be detected using a photometric, a spectrophotometric, a colorimetric, a chemiluminescent, a fluorescent, a radioactive, a mass spectrometric, an electrophoretic, a magnetic resonance imaging, and combinations thereof. The thioaptamer may be selected by synthesizing a random phosphodiester oligonucleotide combinatorial library using a mix of four nucleotides, wherein at least a portion of at least one of the nucleotides in the mix is thiophosphate-modified, to form a thioaptamer combinatorial library, contacting the thioaptamer combinatorial library with the target molecule and isolating the subset of thioaptamers that bind specifically to the target molecule.

The physiological pathway that may be studied using the present invention include: the biosynthesis of cofactors prosthetic groups and carriers (lipoate synthesis, riboflavin synthesis pyridine nucleotide synthesis); the biosynthesis of the cell envelopes (membranes, lipoproteins, glycoproteins, porins, surface polysaccharides, lipopolysaccharides, antigens and surface structures); cellular processes including cell division, chaperones, detoxification, protein secretion, central intermediary metabolism (energy production of phosphorus compounds); energy metabolism including aerobic, anaerobic, ATP proton motive force interconversions, electron transport, glycolysis triose phosphate pathway, pyruvate dehydrogenase, sugar metabolism; purine, pyrimidine nucleotide synthesis, 2′-deoxyribonucleotide synthesis, nucleotide and nucleoside interconversion, salvage of nucleoside and nucleotides, sugar-nucleotide biosynthesis and conversion; regulatory functions including transcriptional and translational controls, DNA replication including degradation of DNA, DNA replication, restriction modification, recombination and repair; transcription including degradation of DNA, DNA-dependent RNA polymerase and transcription factors; RNA processing; translation including amino acyl tRNA synthetases, degradation of peptides and glycopeptides, protein modification, ribosome synthesis and modification, tRNA modification; translation factors transport and binding proteins including amino acid, peptide, amine carbohydrate, organic alcohol, organic acid and cation transport; and other systems for the adaptation, specific function or survival of an artificial organism and combinations thereof. In one specific example, the physiological pathway is cytokine release, e.g., IL-1 through IL-15. One example of a target molecule includes nuclear regulatory factors, e.g., transcriptional activation factors, e.g., NF-IL6, NF-κB, AP-1 and combinations of subunits thereof.

Another embodiment of the present invention is a method of testing a chemical agent that includes the steps of; exposing a cell having a measurable biochemical activity to a thioaptamer that modifies the activity of a target molecule; contacting at least a portion of the cell with the chemical agent and measuring the effect that the chemical agent has on the measurable biochemical activity. The thioaptamer may include SEQ ID NOS: 1-9.

Examples of cells, portions of cells, cell fragments and the like include normal or wild-type cells, which may be one or more cells primary or secondary cells grown in cultures, transformed cells and the like. The cells may be isolated from blood, bodily fluids or secretions or even a portion of a complete tissue, organ, organ system or even a whole organism. Alternatively, the one or more cell may come from an animal knock-out, knock-in, transgenic, over-expressing transgenic, under-expressing-transgenic, conditional knockout, mutant, chimera or combinations thereof.

The present invention also includes a system for testing the biological effects of a molecule in which at least a portion of a cell that includes a target molecule and a measurable biochemical activity is mixed with a thioaptamer that modifies the activity of the target molecule that is added to the cell and a biochemical activity detection device that records differences in the measurable biochemical activity of the cell upon exposure to the molecule. The biochemical activity detection device may include, e.g., one or more chemical and/or biological assays or even a photometric, a spectrophotometric, a colorimetric, a chemiluminescent, a fluorescent, a radioactive, a magnetic resonance imager, a mass spectrometric, a electrophoretic, nuclear magnetic resonance detection system and combinations thereof.

According to one embodiment of the present invention, the thioaptamer is selected by the steps of (a) synthesizing a random phosphodiester oligonucleotide combinatorial library wherein constituent oligonucleotides comprise at least a set of 5′ and 3′ PCR primer nucleotide sequences flanking a randomized nucleotide sequence, (b) amplifying the library enzymatically using a mix of four nucleotides, wherein at least a portion of at least one of the nucleotides in the mix is thiophosphate-modified, to form a thioaptamer combinatorial library, (c) contacting the thioaptamer combinatorial library with a target molecule and isolating a subset of oligonucleotides binding to the target molecule, (d) amplifying the subset of binding thioaptamers enzymatically using a mix of four nucleotides, wherein at least a portion of the thioaptamer is thiophosphate-modified, to form a thioaptamer sub-library and (e) repeating steps (c)-(e) iteratively with increased stringency of the contacting step between each iteration until at least one thioaptamer of defined sequence is obtained.

According to one embodiment of the present invention, no more than three adjacent phosphate sites of the thioaptamers are replaced with phosphorothioate groups. According to another embodiment of the present invention, at least a portion of non-adjacent dA, dC, dG, or dT phosphate sites of the thioaptamer are replaced with phosphorothioate groups. According to yet another embodiment of the present invention, all of the non-adjacent dA, dC, dG, or dT phosphate sites of the thioaptamer are replaced with phosphorothioate groups. According to yet another embodiment of the present invention, all of the non-adjacent dA, dC, dG, and dT phosphate sites of the thioaptamer are replaced with phosphorothioate groups.

The unique chemical diversity of the thioaptamers generated by methodologies provided herein stems from both the nucleotide base-sequence and phosphorothioate backbone sequence. The present method provides enantiomerically pure oligonucleotide products whether the amplification substrates are enzymatically synthesized monothiophosphates or chemically synthesized dithiophosphates. The present thioaptamer methodology provides compounds that are an improvement over existing antisense or “decoy” oligonucleotides because of their stereochemical purity. Chemically synthesized phosphorothioates may be a diastereomeric mixture with 2ⁿ stereoisomers with n being the number of nucleotides in the molecule. These preparations are unsuitable for use in humans because only a small fraction of the stereoisomers will have useful activity and the remaining could have potential adverse effects. In contrast, enzymatically synthesized oligonucleotides are stereochemically pure due to the chirality of polymerase active sites. Inversion of configuration is believed to proceed from Rₚ to Sₚ during incorporation of dNMPαS into the DNA chain. The present dithiophosphate aptamers are free from diastereomeric mixtures.

The present inventors recognized that it is not possible to simply replace thiophosphates in a sequence that was selected for binding with a normal phosphate ester backbone oligonucleotide. Simple substitution was not practicable because the thiophosphates can significantly decrease (or increase) the specificity and/or affinity of the selected ligand for the target. It was also recognized that thiosubstitution leads to a dramatic change in the structure of the aptamer and hence alters its overall binding affinity. The sequences that were thioselected according to the present methodology, using as examples of DNA binding proteins NF-IL6, NF-κB and AP-1 were different from those obtained by normal phosphate ester combinatorial selection.

The present invention takes advantage of the “stickiness” of thio- and dithio-phosphate ODN agents (thioaptamers) to enhance the affinity and specificity to a target molecule. In addition to increased nuclease resistance, the thioaptamers are optimized to decrease non-specific binding to non-target proteins and enhance only the specific favorable interactions with the target to affect a physiological pathway.

In one embodiment of the present invention, a method of identifying therapeutic potential of the thioaptamer is provided by selective modification (increased or decreased activity) of the target protein, upstream or even downstream effects within a physiological pathway. In combination with other compounds that affect a particular physiological pathway, the thioaptamers may be used to dissect the functional activity of one or more target proteins within a cell. The controlled thiolation methodology of the present invention is applicable to the design of specific, nuclease resistant aptamers to virtually any target, including but not limited to, amino acids, peptides, polypeptides, glycoproteins, carbohydrates, nucleotides and derivatives thereof, cofactors, antibiotics, toxins, and small organic molecules, including, e.g., dyes, theophylline and dopamine. It is contemplated, and within the scope of this invention, that the thioaptamers encompass further modifications to increase stability and specificity including, for example, disulfide crosslinking. It is further contemplated and within the scope of this invention that the thioaptamers encompass further modifications including, for example, radiolabeling and/or conjugation with reporter groups, such as biotin or fluorescein, or other functional groups for use in in vitro and in vivo diagnostics and therapeutics.

The present invention further provides the application of this methodology to the generation of novel thiolated aptamers specific for nuclear transcriptional factors, e.g., NF-IL6, NF-κB, AP-1 and/or combinations of subunits thereof. The thioaptamers disclosed herein may be used to affect a wide variety of intracellular signaling events, e.g., NF-κB activation leads to enhanced transcription of a variety of proinflammatory mediator genes, including tumor necrosis factor α, interleukin-1, and inducible nitric oxide synthase. These secreted mediators in turn lead to increased adhesion molecule expression on leukocytes and endothelial cells, increased tissue factor expression on monocytes and endothelial cells, promoting coagulation, vasodilatation, capillary leakiness and myocardial suppression. One or more of these pathways may be isolated and studied using the present invention.

The thioaptamers for use with the present invention may be double or single-stranded DNA, RNA and biochemical variants thereof. The transcription factor may be, e.g., NF-κB, RBP-Jκ, AP-1, NF IL-6, SP-1, GRE, SRE, mixtures thereof and the like. The thioaptamer will generally bind specifically to a protein, e.g., a transcription factor and may also include one or more of the aptamers of SEQ ID NOS.: 1, 2, 3, 4, 5, 6, 7, 8 and 9, e.g.,

| | | |
|---|---|---|
| XBY-6: | 5′-CCAGGAGAT_{S2}T_{S2}CCAC-3′ | SEQ ID NO.: 1 |
| | 3′-GG_{S2}TCC_{S2}TC_{S2}TAAGG_{S2}TG-5′ | |
| XBY-S2: | 5′-CCAGT_{S2}GACT_{S2}CAGT_{S2}G-3′ | SEQ ID NO.: 2 |
| | 3′-GG_{S2}TCAC_{S2}TGAG_{S2}TCAC-5′ | |
| XBY-S1: | 5′-T_{S2}T_{S2}GCGCGCAACAT_{S2}G-3′ | SEQ ID NO.: 3 |
| | 3′-AACGCGCG_{S2}T_{S2}TG_{S2}TAC-5′ | |
| XBY-C2: | 5′-CCAGTGACTCAGTG-3′ | SEQ ID NO.: 4 |
| | 3′-GGTCACTGAGTCAC-5′ | |
| XBY-C1: | 5′-TTGCGCGCAACATG-3′ | SEQ ID NO.: 5 |
| | 3′-AACGCGCGTTGTAC-5′ | |
| | 5′-tGTGcAGGGACTgAtGaCGGt-3′, | SEQ ID NO.: 6 |
| | 5′-CtGTGCatCGAaGTTtGCAtTt-3′, | SEQ ID NO.: 7 |
| | 5′-AtGcAcAtCtCaGgAtGaCGGt-3′, | SEQ ID NO.: 8 |
| | 5′-AGTTGcAGGtCaGgACCCAtTt-3′, | SEQ ID NO.: 9 |

wherein the lowercase letters represent the thiophosphate 3′ to the base. In one examples, the method of treatment may be directed to a neuropathologic viral infection and include the steps of identifying a patient suspected of being infected with a neuropathologic virus; and providing the patient with a therapeutic amount of a partially thioaptamer specific for transcription factor involved in immune cell activation. A thioaptamer for use in the method of treatment may be XBY-S2.

The thioaptamers and method of the present invention may be used, e.g., to effect and detect biochemical and/or physiological changes in the immune response after challenge with an antigen (e.g., a pathogen or portions thereof). The physiological read-out may be, e.g., the activation or deactivation of the innate immune response and/or modifications to the type of adaptive immune response mounted (humoral versus cell-based). One example of a physiological read-out is, e.g., a change in the type of helper T cell involved with or direct the immune response toward a cellular- or humoral-based immune response. The thioaptamers may be used to modify the timing and amount of cytokines released, e.g., interleukin-1 (IL-1), interleukin-2 (IL-2), interleukin-3 (IL-3), interleukin-4 (IL-4), interleukin-5 (IL-5), interleukin-6 (IL-6), interleukin-7 (IL-7), interleukin-8 (IL-8), interleukin-10 (IL-10), interleukin-11 (IL-11), interleukin-12 (IL-12), interleukin-13 (IL-13), interleukin-14 (IL-14), interleukin-15 (IL-15), or other interleukins, Type I Interferon, Type II Interferon, tumor necrosis factor alpha (TNF-alpha), transforming growth factor-beta (TGF-beta), lymphotoxin migration inhibition factor, granulocyte-macrophage colony-stimulating factor (GM-CSF), monocyte-macrophage CSF, granulocyte CSF, vascular epithelial growth factor (VEGF), angiogenin, transforming growth factor (TGF-alpha), fibroblast growth factor, angiostatin, endostatin, mixtures or combinations thereof.

For example, the thioaptamer may affect the stimulation of specialized antigen presenting cells (APCs), e.g., macrophages, dendritic cells and B cells or non-specialized immune or even non-immune cells. Alternatively, the thioaptamer may activate an innate immune response, e.g., through Toll-Like receptors that stimulate lymphocytes such as APCs, B cells and T cells. In one example, the aptamer activates an innate immune response that includes the simultaneous activation of macrophages and dendritic cells and of B cells and T cells.

### BRIEF DESCRIPTION OF THE DRAWINGS

For a more complete understanding of the features and advantages of the present invention, reference is now made to the detailed description of the invention along with the accompanying figures and in which:

FIG. 1 is a graph that shows the production of TNF-α in P388D1 cells. Cells were treated with polyI/C (25 μg/ml) and media samples were taken at indicated times, the TNF-α levels in the media were determined using commercially available ELISA;

FIGS. 2A, 2B and 2C are bar graphs that show the production of by P388D1 cells infected with Pichinde P2 or Pichinde P18 taken three days post-infection and assayed for TNF-α (3A), IL-6 (3B) and IL-12 (3C);

FIG. 3 is a graph that shows the secretion of TNFα as measured by ELISA of Mouse P388D1 macrophage cultures that were treated with XBY-S2 for 12 hours followed by stimulation with PolyI/C and harvested at 24 hrs;

FIG. 4 is a graph of IL-6 production assayed by ELISA of mouse P388D1 macrophage cultures treated with XBY-S2 for 12 hours followed by stimulation with PolyI/C and harvested at 24 hrs;

FIG. 5 is a graph that shows survival curves following Pichinde P18 infection in guinea pigs treated with the NF-κB thioaptamer, XBY-6, the scrambled control, B92, or vehicle, MT, of animals infected by injection of 1000 plaque forming units (pfu) of Pichinde P18 at day 0, treatment was by intraperitoneal injections at days 0, 1 and 2;

FIG. 6 is a graph that shows survival curves of guinea pigs with thioaptamers for infection by arenavirus;

FIG. 7 is a graph that shows survival curves following West Nile Virus infection in guinea pigs treated with the NF-κB aptamer X BY-6, the AP-1 aptamer XBY-S2, or the liposome vehicle of animals infected by injection with lethal doses of West Nile Virus;

FIG. 8 are graphs that show SELDI detection of recombinant p50 using Epoxy-activated ProteinChip Arrays with XBY-6 (top), IgκB 22-mer duplex (middle) or control, poly (dLdC) (bottom) covalently linked to surfaces;

FIG. 9 are graphs that show the detection of recombinant p50 on gel beads using XBY-6. The top two SELDI MS extract from beads spotted onto a NP20 ProteinChip. The bottom two SELDI spectra taken on beads themselves, in which the control is no XBY-6 covalently attached to beads with aminolinker; and

FIG. 10 is a graph that shows the SELDI MS capture of endogenous p50 (p105) from nuclear extracts on Ciphergen PS20 Proteinchip Arrays, the topgraph shows covalently linked XBY-6 to array surface, in the bottom, control no XBY-6 linked to surface.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, “synthesizing” of a random combinatorial library refers to chemical methods known in the art of generating a desired sequence of nucleotides including where the desired sequence is random. Typically in the art, such sequences are produced in automated DNA synthesizers programmed to the desired sequence. Such programming can include combinations of defined sequences and random nucleotides.

“Random combinatorial oligonucleotide library” means a large number of oligonucleotides of different sequence where the insertion of a given base at given place in the sequence is random. “PCR primer nucleotide sequence” refers to a defined sequence of nucleotides forming an oligonucleotide which is used to anneal to a homologous or closely related sequence in order form the double strand required to initiate elongation using a polymerase enzyme. “Amplifying” means duplicating a sequence one or more times. Relative to a library, amplifying refers to en masse duplication of at least a majority of individual members of the library.

As used herein, “thiophosphate” or “phosphorothioate” are used interchangeably to refer to analogues of DNA or RNA having sulphur in place of one or more of the non bridging oxygens bound to the phosphorus. Monothiophosphates or phosphoromonothioates [αS] have only one sulfur and are thus chiral around the phosphorus center. Dithiophosphates are substituted at both oxygens and are thus achiral. Phosphoromonothioate nucleotides are commercially available or can be synthesized by several different methods known in the art. Chemistry for synthesis of the phosphorodithioates has been developed by one of the present inventors as set forth in U.S. Pat. No. 5,218,088 (issued to Gorenstein, D. G. and Farschtschi, N., Jun. 8, 1993 for a Process for Preparing Dithiophosphate Oligonucleotide Analogs via Nucleoside Thiophosphoramidite Intermediates), relevant portions incorporated herein by reference.

As used herein, the terms “thio-modified aptamer” and “thioaptamer” are used interchangeably to describe oligonucleotides (ODNs) (or libraries of thioaptamers) in which one or more of the four constituent nucleotide bases of an oligonucleotide are analogues or esters of nucleotides that normally form the DNA or RNA backbones and wherein such modification confers increased nuclease resistance; and the DNA or RNA may be single or double stranded. For example, the modified nucleotide thioaptamer can include one or more phosphorothioate or phosphordithioate linkages selected from wherein the group: dATP(αS), dTTP(αS), dCTP(αS), dGTP(αS), rUTP (αS), rATP(αS), rCTP(αS), rGTP(αS), dATP(αS₂), dTTP(αS₂), dCTP(αS₂), dGTP(αS₂), rATP(αS₂), rCTP(αS₂), rGTP(αS₂) and rUTP(αS₂) or modifications or mixtures thereof. In another example, no more than three adjacent phosphate sites of the modified nucleotide aptamer are replaced with phosphorothioate groups. In yet another example, at least a portion of non-adjacent dA, dC, dG, dU or dT phosphate sites of the modified nucleotide aptamer are replaced with phosphorothioate groups. In another example of a thioaptamer, all of the non-adjacent dA, dC, dG, or dT phosphate sites of the modified nucleotide aptamer are replaced with phosphorothioate groups; all of the non-adjacent dA, dC, dG, and dT phosphate sites of the modified nucleotide aptamer are replaced with phosphorothioate groups; or substantially all non-adjacent phosphate sites of the modified nucleotide aptamer are replaced with phosphorothioate groups. In still another embodiment of the present invention, no more than three adjacent phosphate sites of the modified nucleotide aptamer are replaced with phosphorodithioate groups. The thioaptamers may be obtained by adding bases enzymatically using a mix of four nucleotides, wherein one or more of the nucleotides are a mix of unmodified and thiophosphate-modified nucleotides, to form a partially thiophosphate-modified thioaptamer library. In another example of “thioaptamers” these are made by adding bases to an oligonucleotide wherein a portion of the phosphate groups are thiophosphate-modified nucleotides, and where no more than three of the four different nucleotides are substituted on the 5′-phosphate positions by 5′-thiophosphates in each synthesized oligonucleotide are thiophosphate-modified nucleotides.

Thiophosphate nucleotides are an example of modified nucleotides. “Phosphodiester oligonucleotide” means a chemically normal (unmodified) RNA or DNA oligonucleotide. Amplifying “enzymatically” refers to duplication of the oligonucleotide using a nucleotide polymerase enzyme such as DNA or RNA polymerase. W here amplification employs repetitive cycles of duplication such as using the “polymerase chain reaction”, the polymerase may be, e.g., a heat stable polymerase, e.g., of Thermus aquaticus or other such polymerases, whether heat stable or not.

“Contacting” in the context of target selection means incubating a oligonucleotide library with target molecules. “Target molecule” means any molecule to which specific aptamer selection is desired. “Essentially homologous” means containing at least either the identified sequence or the identified sequence with one nucleotide substitution. “Isolating” in the context of target selection means separation of oligonucleotide/target complexes, preferably DNA/protein complexes, under conditions in which weak binding oligonucleotides are eliminated.

By “split synthesis” it is meant that each unique member of the combinatorial library is attached to a separate support bead on a two (or more) column DNA synthesizer, a different thiophosphoramidite or phosphoramidite is first added onto both identical supports (at the appropriate sequence position) on each column. After the normal cycle of oxidation (or sulfurization) and blocking (which introduces the phosphate, monothiophosphate or dithiophosphate linkage at this position), the support beads are removed from the columns, mixed together and the mixture reintroduced into both columns. Synthesis may proceed with further iterations of mixing or with distinct nucleotide addition.

Aptamers may be defined as nucleic acid molecules that have been selected from random or unmodified oligonucleotides (“ODN”) libraries by their ability to bind to specific targets or “ligands.” An iterative process of in vitro selection may be used to enrich the library for species with high affinity to the target. The iterative process involves repetitive cycles of incubation of the library with a desired target, separation of free oligonucleotides from those bound to the target and amplification of the bound ODN subset using the polymerase chain reaction (“PCR”). The penultimate result is a sub-population of sequences having high affinity for the target. The sub-population may then be subcloned to sample and preserve the selected DNA sequences. These “lead compounds” are studied in further detail to elucidate the mechanism of interaction with the target.

Thioaptamers and other nucleic acid analogs (e.g. peptide nucleic acids (PNAs), methylphosphonates, etc.) are emerging as important agents in therapeutics, drug discovery and diagnostics. Three key attributes define the unique ability of (thio)aptamers to perform their essential functions: (1) they target specific proteins in physiological pathways; (2) their sequence and structure is not intuitively obvious from canonical biologics and oftentimes can only be deduced by combinatorial selection against their targets; and (3) they bind their targets with higher affinities than do naturally occurring nucleic acid substrates. Importantly, the backbone modifications of thioaptamers and their nucleic acid backbone analogs enable aptamers to be introduced directly into living systems with in vivo lifetimes many times greater than unmodified nucleic acids, due to their inherent nuclease resistance of the modified aptamers. The inherent nuclease resistance is extraordinarily important for their efficacy in use.

As important as aptamers obviously are to the discovery of new therapeutics and diagnostics, the present inventors have discovered a significant new use for them that has not been previously disclosed. With the advent of the description of the human genome and the emergence of high throughput analytical techniques such as genomic microarrays and proteomics gels and chips, a vast wealth of data on the functions of biological systems is quickly emerging. A result of the genome project is that a major paradigm shift in biological and biomedical discovery will continue and expand over many future decades, if not centuries. The impact on human health and the management of disease is almost incalculable.

Central to this revolution in health science is the discovery of detail of the pathways through which physiological systems communicate. This is vitally important to the understanding of the power of aptamers because each individual aptamer targets a specific node in a physiological pathway. Indeed, the patents and publications of Gorenstein, et al. describe in detail the ability of certain combinatorially selected aptamers to target specific elements in the signalling pathways controlled by the master transcriptional regulators of the NF-κB homo- and heterodimer family, with very high affinity. By understanding how biological pathways operate and interconnect, biomedical researchers can learn how to target specific nodes (often proteins) in these pathways, in order to modulate their activity through the use of drugs. The power of this knowledge is enormous and will be the enabling lever through which human pain and suffering due to disease, aging and other physiological circumstances is managed and alleviated in future decades through molecular medicine.

Unfortunately, heretofore it has not been intuitively obvious as to how biomedical research might learn to understand the exceedingly complex physiological pathways in sufficient detail to identify targets for therapy. Indeed, though the model of how this might be done is well understood through innovations in systems engineering and computational technology, the ability to probe these systems at a sufficient level of complexity to be practically useful has not existed. Simple organisms lend themselves to the genetic engineering necessary to construct a sufficient number of genetic variants (hundreds to thousands) so that scientists can probe the putative pathways through which these organisms maintain homeostasis, respond to diseases, and sustain life. Such simple systems rarely shed much light on how more complex organisms operate, at a level sufficient to support drug discovery.

Typically, the systems studied have been either prokaryotic or exceedingly simple single cell eukaryotic organisms (e.g. yeast), that are insufficiently complex to aid human biomedical research in a practical way. The major barrier to such research on complex, multi-cellular systems has been the technological and ethical barriers to genetically engineering large numbers of groups containing known genetic mutations at specific loci. Oftentimes, when attempted in lower species such as mice, these “knockouts” are lethal and the effects of the knockout are so diverse as to be only moderately informative. The long-term value of such genetically engineered animal models is clearly limited, and obviously not applicable to humans.

Human pathway and systems research, however, cannot possibly progress beyond its current infancy without the ability to perturb those essential homeostatic pathways that define the states of health, infirmity and disease. It is intuitively obvious to anyone knowledgeable in the science or systems engineering and systems analysis that without the ability to probe and perturb specific nodes within the system, one can never learn in detail the operations and operational parameters of a complex system. The simple fact is that genetic “knockouts” will never be feasible for higher species in sufficiently large numbers to make progress in human pathways and systems science.

Aptamers, however, clearly present us with an enabling mechanism to achieve our goals in the elucidation of human physiological pathways. By a major definition of use, an aptamer is specifically targetted to a node (perhaps a protein) in a physiological pathway, which is one practical defining feature of its utility as a therapeutic or diagnostic. This feature, indeed, enables the present invention. By constructing an aptamer to a specific protein (e.g. NF-κB p50 homodimer), one has achieved the optimum tool to perturb the pathway(s) that this protein participates in, in a controlled and systematic fashion. One can vary the dose and timing (in multiple doses) of the aptamers easily, thereby allowing measurement of changes in the “state” of the organism (or in an in vitro system) via high throughput techniques such as gene microarrays, proteomic microarrays and 2D-gels commonly used in proteomics. Other large-scale techniques should be obvious to anyone familiar with this art.

The “one aptamer-one protein” dogma is central to the success of the method. By targetting one protein or “node” in the pathway, the systematic study is enabled. The technology of how this works and how these data are subsequently deconvoluted is well described. Essentially, in any complex system with interacting elements and states, its collective behavior can be described in terms of abundance (e.g. concentration or mass) of any single entity (node) at a given time, and that entity's time rate of change to any other entity via a connecting pathway. There is thus, a canonical set of parameters that describe any given (pseudo)static state at any given time. It is well-known in the physical sciences (e.g. spectroscopic methods such as NMR) that by perturbing a static state through some specific agent (photons in NMR), all the internal parameters that describe that state are encoded in its relaxation back to equilibrium. One only has to deconvolute these data to discover all the interacting parameters that define that state or pathway.

The essential perturbing element in complex physiological pathways that are central to biomedical research is thus the aptamer. One introduces a measured dose of an aptamer selected for a specific node in a physiological pathway and then measures the activity of the genes (through the abundance of the mRNA produced) on a gene microarray and/or the abundance of the gene products (e.g. proteins) via 2D gels or proteomic arrays or multicolumn chromatography or whatever technique is deemed most useful by the investigator. The patterns within the data collected describe the behavior of the pathway with respect to the node selected. One can in turn select many different nodes to target with aptamers, and could conceivably target more than one node at a time. The investigator is only limited by the ability to deconvolute the resulting data. It is especially important to point out that it is not necessary to a priori know each of the important nodes in a system. The gathering of significant amounts of data on such a large scale will enable a putative model to be advanced to a higher level through the discovery of heretofore-unknown members or elements that will be indicated by detailed analysis of the data. Thus, the process becomes increasingly more fruitful as a learned investigator iterates through succeeding generations of experimentation, suggested by the preceding descriptions of the path or state.

It is important to note that the use of aptamers in this fashion is far from obvious, even to investigators versed in this art. Historically, the sole means of investigating physiological pathways has involved the use of genetic knockouts and/or varying experimental parameters such as temperature. These methods are limited to simple prokaryotes or primitive eukaryotes and are not feasible for higher, more complex species such as mice and humans, and no other perturbational method or agent has been heretofore suggested. Thus, aptamers are enabling agents for the science and technology of the discovery of the behavior and makeup of physiological systems, especially for the purpose of developing drugs and other therapeutics for the defeat of disease or infirmity, and the promotion and maintenance of health. It is difficult to find an analogy in science or technology to describe the importance of this invention as an enabling agent in such an important area of discovery. The most cogent recent example that occurs to us is the use of polymerase fro Thermus aquaticus as the enabling reagent in PCR. It would be hard to underestimate the importance of PCR to modern molecular biology or biomedicine, and importantly, the development of the aptamer for use in physiological pathway elucidation may be equally as important.

The portions of cells, cells, tissues, organs and/or animals that may be used with the present invention may be: wild-type, chimeric, transgenic, knock-outs, knock-ins or combinations thereof. As used herein, the terms “normal” and “wild-type” are intended to be synonymous, and to denote any nucleotide sequence typically found in nature. The terms “mutation” or “mutated” as used herein are intended to denote an alteration in the “normal” or “wild-type” nucleotide sequence of any nucleotide sequence or region of the allele. An allele of a gene is said to be mutated if: (1) it is not expressed in a cell or animal; (2) the expression of the allele is altered with respect to the expression of the normal allele of the gene; and/or (3) the allele expresses a gene product, but that gene product has altered structure, activity or characteristics relative to the gene product of a normal allele of that gene.

The terms “mutated” and “normal” are defined relative to one another; where a cell has two chromosomal alleles of a gene that differ in nucleotide sequence, at least one of these alleles is a “mutant” allele as that term is used herein. Also, there may be naturally occurring genetic variation in cellular adhesion genes in mouse or human, and these naturally occurring variations may affect inflammatory processes.

As used herein, a “chimeric” animal differs from a “transgenic” animal in that a chimeric animal's cells only contain and express the introduced gene sequence, whereas other cells have been unaltered. Chimeric animals transmit the introduced gene sequence to its progeny only if the introduced gene sequences are present in the germ cells of the animal. In contrast, all of the cells of a “transgenic” animal contain the introduced gene sequence. Therefore, every transgenic animal is capable of transmitting the introduced gene sequence to its progeny.

Yet another source for cells may be an animal modified by homologous recombination, e.g., knock-outs, knock-ins, and the like which may even by conditional in nature. As used herein, an allele is said to be “homologous” if its introduction alters or replaces one of the two alleles of a gene in a cell. Thus, homologous recombination involves a sequence-specific process by which cells transfer a “region” of DNA from one DNA molecule to another. As used herein, a “region” of DNA refers generally to any nucleic acid molecule, from a single base to a substantial fragment of a chromosome.

As used herein the terms “detectable activity” or a “measurable biochemical activity” are used to define one or more assays that provide a result that may be compared to the prior state of the system. Examples of detectable activities include: proliferation; apoptosis; one or more enzymatic activities; small molecule, protein, lipid carbohydrate amino acid and/or nucleic acid use, destruction, production, release, modification and the like. These activities may be measured in, e.g., whole cells, tissues, organs, whole animals or portions thereof. Methods of detection include, e.g.: chemical and/or biological assay, photometric, spectrophotometric, calorimetric, chemiluminescent, fluorescent, radioactive, mass spectrometric, electrophoretic, nuclear magnetic resonance and/or combinations thereof. Examples of physiological pathways for which modifications in activity may be measured include, e.g.: biosynthesis of cofactors prosthetic groups and carriers (lipoate synthesis, riboflavin synthesis pyridine nucleotide synthesis); the biosynthesis of the cell envelopes (membranes, lipoproteins, porins, surface polysaccharides, lipopolysaccharides, antigens and surface structures); cellular processes including cell division, chaperones, detoxification, protein secretion, central intermediary metabolism (energy production of phosphorus compounds); energy metabolism including aerobic, anaerobic, ATP proton motive force interconversions, electron transport, glycolysis triose phosphate pathway, pyruvate dehydrogenase, sugar metabolism; purine, pyrinidine nucleotide synthesis, 2′-deoxyribonucleotide synthesis, nucleotide and nucleoside interconversion, salvage of nucleoside and nucleotides, sugar-nucleotide biosynthesis and conversion; regulatory functions including transcriptional and translational controls, DNA replication including degradation of DNA, DNA replication, restriction modification, recombination and repair; transcription including degradation of DNA, DNA-dependent RNA polymerase and transcription factors; RNA processing; translation including amino acyl tRNA synthetases, degradation of peptides and glycopeptides, protein modification, ribosome synthesis and modification, tRNA modification; translation factors transport and binding proteins including amino acid, peptide, amine carbohydrate, organic alcohol, organic acid and cation transport; and other systems for the adaptation, specific function or survival of an artificial organism and combinations thereof.

The following examples are presented to further illustrate the present invention and are not to be construed as unduly limiting the scope of the present invention.

NF-κB dithioate modified aptamers. According to the literature, complete thioation of the CK-1 (or CK-14) aptamer provides an effective agent capable of specifically binding various NF-κB/Rel dimers. The present inventors have found this not to be the case. Because of the specificity of the interaction between the thioated phosphates and the protein, CK-14 14-mer duplexes with strategically placed dithioate linkages were synthesized. According to an embodiment of this invention, the substitutions were very significant. They resulted in altered binding specificity, and a lack of the extreme “stickiness” of the fully thioated aptamer. For example, when only one or two dithioate linkages were placed in a molecule, the inhibition/binding of the oligonucleotide to recombinant protein was similar to that of the unsubstituted aptamer.

It was found that XBY-6 shifts a complex in nuclear extracts from 70Z/3 cells. By using specific antibodies to supershift the complex, p50 was identified as one component of the complex, and may be the p50/p50 dimer. Only one major band was seen, however, even though the lysate contains at least two major distinguishable NF-κB complexes (p50 homodimers and p50/p65 heterodimers). These data show that by substituting only a limited number of internucleoside linkages, the binding specificity can be altered. By using an aptamer that distinguishes among various NF-κB dimers within the cell, this aptamer was used to bind to and monitor a single NF-κB complex in cell extracts, and on a substrate chip. The same aptamer can also inactivate a single NF-κB dimer within a cell. These functions point to the importance of not only structure-based design, but also the thiophosphate combinatorial selection protocols to identify minimally substituted thioated oligonucleotides with high affinity, high binding specificity and increased nuclease resistance in vitro and in vivo.

As the substitutions of dithiophosphate were increased, binding by the [S₂]-ODN oligonucleotide increased dramatically. For example, in a standard competitive binding assay, ³²P IgκB promoter element ODN is incubated with recombinant p65 and varying amounts of XBY aptamer competitor. The relative binding ability of the unlabeled ODNs is determined by the concentration needed to effectively compete with the standard labeled ODN. XBY1 through 6 correspond to CK-14 aptamers with 1 through 6 dithiophosphate substitutions, respectively. The present inventors, according to an embodiment of this invention, developed an oligonucleotide containing six dithioate linkages on the two strands, termed XBY-6. Unlike the fully substituted [S]-ODN CK-14, the XBY-6 hybrid backbone [S₂]-ODN aptamers bound more tightly to p50/p50 (5-fold) than to the p65 homodimer. Additionally, the XBY-6 aptamer also bound a single NF-κB dimer in cell extracts, while the standard phosphodiester ODN showed no NF-κB-specific binding in extracts.

Enhanced affinity of the dithioate aptamers for the NF-κB dimers may correlate with proximity of the modified phosphate to a group in the binding site (largely a basic amino acid side chain). It follows that the greater the number of such interactions, the greater the affinity. Based upon the crystal structures of duplex sites bound to various NF-κB dimers, a number of phosphates are in close contact with groups on the NF-κB dimers. For XBY-6, proposed contacts shown are based on modeling. Note that p50 homodimers have contacts to the right hand side TpTpC phosphates whereas in the p65 homodimer, these contacts are missing. In cell culture, XBY-6 appears to bind to a p50 homodimer, consistent with modeling results. A 1:1 binding stoichiometry of p65 to the 22-mer binding site known as IgκB and a Kd near 4 nM. The dithiophosphate aptamer, XBY6, has been found to have a binding affinity to p65 homodimer of 1.4 nM, and sub-nM to the p50 homodimer.

Additional dithiophosphate modified CK-14 aptamers were synthesized to take advantage of the putative differential effects for dithioate interactions and stabilization of the complexes. The results confirmed that affinity was highest for those dithioate aptamers containing the greatest number of favorable phosphate contacts to the specific dimer, as based upon the modeling.

Thioselection of Phosphorodithioate Aptamers Binding to NF-κB (p65-p65). Library Generation. A random combinatorial library of normal phosphoryl backbone oligonucleotides was synthesized by an automated DNA synthesizer that was programmed to include all 4 monomer bases of the oligonucleotide during the coupling of residues in a randomized segment. A 62-mer has been constructed with a 22 base pair random central segment flanked by 19 and 21 base pair PCR primer regions: 5′dATGCTTCCACGAGCCTTTC(N22)CTGCGAGGCGGTAGTCTATTC3′ (SEQ ID NO.: 10). The resulting library thus exists as a population with potentially 4²² (10¹³) different possible sequences.

Thiophosphate substitution and selection. For example, when modifying a portion of, e.g., dATP in the duplex oligonucleotide library with phosphoromonothioate backbone substituted at dA positions only, the sample was then synthesized by PCR amplification of the 62-mer template using commercially available Taq polymerase and using a mix of dATP and dATP(αS); dTTP; dGTP and dCTP as substrates. Likewise, the other bases may also be partially be thio-modified using at least a portion of the bases as thiophosphate substituted bases using: dATP(αS), dTTP(αS), dGTP(αS) and/or dCTP(αS) as substrates. As will be appreciated by those of skill in the art, any of the nucleotides may be the one or more nucleotides that is selected to have the thiol modification.

The random library was screened to identify sequences that have affinity to the p65 homodimer. PCR amplification of the single stranded library provides chiral duplex phosphorothioate 62-mer at all dA positions other than the primers. The amplification product was then incubated with the p65 dimer for 10 minutes at 25° C. and filtered through pre-soaked Millipore HAWP 25 mm nitrocellulose filters. The combinatorial thiophosphate duplex library was screened successfully for binding to the p65 dimer. The filter binding method was modified to minimize non-specific binding of the thiophosphate oligonucleotides to the nitrocellulose filter.

The thiophosphate substituted DNA was eluted from the filter under high salt and under protein denaturing conditions as will be known to those of skill in the art. Subsequent ethanol precipitation and another PCR thiophosphate amplification provide product pools for additional rounds of selection. In order to increase the stringency of binding of the remaining pool of DNA in the library and select tighter binding members of the library, the KCl concentration was increased in subsequent rounds from 50 to 200 mM. The stringency of selection was also manipulated by increasing the volume of washing solution as the number of iterations are increased. A negative control without protein was performed simultaneously to monitor any non-specific binding of the thiophosphate DNA library to the nitrocellulose filter.

Thioselection against the p65-p65 of NF-κB was carried through 10 rounds. Cloning and sequencing according to standard methods known to those in the art was performed after 10 iterations had been completed. The sequences were lined up by either their 5′-3′ o r 3′-5′ ends choosing the G rich strand, thus finding a consensus pattern in the sequences. The sequence obtained for a 22-nucleotide variable region in which all dAs were thiolated, is an example of a thioaptamer that shows a conserved consensus site containing two tandem decameric κB motifs separated by G*. A general consensus site for the 22-nt variable region of a new combinatorial library was identified which binds tightly to NF-κB:

| | |
|---|---|
| GGGCG T ATAT G* TGTG GCGGG GG. | (SEQ ID NO.: 11) |

Surprisingly, this sequence differs from the CK-1 sequence of 14 bases. The GGGCG is conserved at both ends of the sequence and finishes with a purine pyrimidine alternation of bases (ATAT or GTGT) centered around the G*. The binding characteristics of this 22-mer suggests that two p65 homodimers bind to the selected sequence and that the p65 homodimers interact in a head to head fashion enhancing their affinity to the mutated DNA.

The two NF-κB dimers bind to the thioselected [S]-ODNs creates a novel invention providing for the development of even more highly selective thiolated aptamers targeted to specific NF-κB/Rel homo- and hetero-dimers, based not only on the protein-DNA contacts, but also on protein-protein contacts. Orientation of each of the NF-κB/Rel dimers on such an aptamer will tightly constrain the optimal dimer-dimer contacts and will presumably differ for each homo- or hetero-dimer. The present invention provides a thioselection methodology that targets any number of different protein-protein complexes, not just those from NF-IL6 and NF-κB/Rel.

Thioselection against NF-κB p65*p65 through 20 rounds was completed and a general consensus site for the 22-nt variable region of a combinatorial library: dGGG GTG NTG TXX XGN GXN XNC; SEQ ID NO. 12 (X=G or C, N=any base) was identified.

The combinatorial libraries that include either: (1) mixed backbone [S₂]-ODN agents made using a split synthesis combinatorial chemistry approach; or (2) combinatorial libraries of [S]-ODN agents made using the enzymatic approach may be used to target specific portions or components of specific physiological pathways. In this example, the thioaptamers and aptamers were used in a proteomics chip to identify a target ODN sequence and modification and/or the protein target within the physiological or biochemical pathway.

Phosphoromonothioate and Phosphorodithioate Aptamers: Split Synthesis Combinatorial Selection. A split and pool synthesis combinatorial chemistry method for creating a combinatorial library of thioated oligonucleotide agents (either monothiophosphate or dithiophosphate) on polyvinylbenzene support was also developed.

Library Construction. A split synthesis combinatorial chemistry method was developed to create a combinatorial library of [S₂]-ODN agents. In this method each unique member of the combinatorial library is attached to a separate support bead. Proteins that bind tightly to only a few of the 10⁴-10⁶ different support beads may be selected by, e.g., deprotecting a single aptamer bead in a 96-well plate in a high-throughput assay (e.g., testing for siRNA activity), or by binding the protein directly to the beads and then identifying which beads have bound protein by immunostaining techniques.

A two column DNA synthesizer (Expedite 8909 DNA synthesizer) was used for library construction. In the first round of solid phase synthesis, a phosphoramidite (for example, C) was coupled to equal portions of the support bead with free hydroxyl functional groups, and after oxidation, the resulting product was a nucleotide (C) bound to the bead support via a phosphotriester linkage. In the second round, a different thiophosphoramidite was added onto both identical supports (at the appropriate sequence position) on each column. (For example, G on column 1, and thioA on column 2). After the normal cycle of S oxidation and blocking (which introduces the dithiophosphate linkage at this position), the support beads were removed from the columns, mixed together and the mixture reintroduced into both columns. At the next randomized position, a thiophosphoramidite with either a different or the same base was then added to each of the columns. Upon mixing, the end products were a mixture of two kinds of bead bound dinucleotides included phosphorotriester and phosphodithiotriester oligonucleotides. Cycles of mixing and separating may be continued for “n” internucleoside phosphorodithioates.

If additional coupling steps and split/pool synthesis were carried out, the end products included a combinatorial library of aptamers with varying phosphorodithioate or normal phosphate esters on the ODNs attached to the support (each bead contained a single sequence with a specified backbone modification that was identified by the base—in the above example any dA at position 2 of the sequence will be a 3′-dithioate since only thioA phosphorothioamidite was used in the second round and a G at position 2 would indicate that it contains a 3′-phosphate).

On completion of the automated synthesis, the column was removed from the synthesizer and dried with argon. The bead-bound, fully protected ODNs were treated with 1 mL of concentrated ammonia for 1 hour at room temperature, incubated at 55° C. oven for 15-16 hours, removed from the oven and cooled to room temperature. The beads were thoroughly washed with double distilled water.

In each run used in this example, no effort was made to use sequence to define the position of the monothioate or dithioate, however, the site of [S₂] or [S] modification could be identified by taking advantage of the difference in chemical reactivity between phosphate and phosphorothioate (and dithioates). The difference in chemical reactivity allows the ODN to be cleaved from the bead at sites of sulfur substitution. The aptamer may be sequenced directly and the location of the thioated internucleoside linkages determined independent of the base sequence. After ³²P-end labeling, the hybrid [S₂]-ODNs were alkylated with agents such as 2-iodoethanol, while normal phosphates were not. Addition of dilute NaOH cleaves only at the thio- (or dithio-) phosphate. Standard sequencing gel electrophoresis could be used to determine the size of the cleaved fragments, and thus the position of the modified phosphate backbone. Importantly, using this coupling scheme with the non-cleavable linker attaching the first phosphoramidite to the bead (provided by Andrew Ellington, UT Austin), the ODNs were still covalently attached to the beads after complete deprotection.

An IgκB 22-mer single strand sequence that is recognized by NF-κB on the non-cleavable linker bead was synthesized. The complementary strand was hybridized to the bead containing the IgκB 22-mer single strand sequence. The longer ODN, with two primer sequences flanking the NF-κB central binding site, can be used for one bead-one aptamer PCR and the ODN sequencing, allowing identification of the one aptamer bound to one selective bead.

Aptamer Selection. In this example, NF-κB target protein was bound to the beads (IgκB site bound bead) and washed at various salt and urea concentrations to remove weakly bound protein. The beads were physically separated from the unstained (unbound) beads. Multicolor flow cytometry and cell sorting could also be used to visualize and sort the protein-bound aptamer beads and select the tightest binding aptamer-protein complexes.

After selection, the bead bound sequence containing both 5′ and 3′ primer sites (the covalently linked aptamer) could be amplified by PCR, and the fragment cloned and sequenced. The IgκB sequence was flanked by 18 base pair PCR primer regions. The upstream primer (5′-ATGCCTACTCGCGAATTC-3′; SEQ ID NO.: 13) contained nucleotide sequences encoding an EcoRI site. The downstream primer (5′-GAACAGGACCACCGGATCC-3′; SEQ ID NO.: 14) contained nucleotide sequences encoding a BamHI site. The single strand IgκB sequence was converted into duplex DNA on the bead in a standard Klenow reaction.

PCR was performed as follows: A reaction mix containing water, DNA polymerase buffer, dNTP mix, downstream primer, DNA polymerase I (Klenow, Promega), and the IgκB aptamer-bead complex was prepared and incubated at 37° C. for 5 hours. The product, containing double-stranded IgκB sequences attached to the beads, was amplified by PCR. PCR products were cloned into a TOPO TA vector (Invitrogen) and sequenced. Automated DNA sequence analysis showed that the sequence was identical to the sequence programmed into the synthesizer. If this were a dithio or monothio modified bead-bound sequence, PCR has also been shown to amplify these templates to identify an oligonucleotide bound to a bead.

Detection and Quantification Scheme. The protein bound to the two-dimensional spotted microarray or bead-based microarray may be visualized using methods known to the art such as commercially available stains, antibodies and reagents. Protogold, a general protein stain with sensitivity to 1 pg, provides a very sensitive colorimetric detection system that may be used to measure the binding of diverse proteins to different ODNs on the same microchip. Alternatively as described above, fluorescent labels may be attached covalently to the proteins in cellular extracts. For differential display, proteins from two different sources may be labeled with two different fluorescent labels. ELISA sandwich methods known to the art with catalyzed reporter deposition for signal amplification or fluorescent-tagged polyclonal antibodies to particular proteins may also be adapted when specific proteins are to be monitored.

Both recombinant proteins and nuclear extracts of cells have been used. In one example, microarrays may be used to detect multiple transcription factor DNA-binding activities on a single chip by using the selected aptamers/thioaptamers specific for a particular NF-κB or NF-IL6 transcription factor, as well as, using the well-established binding sites for other cellular transcription factors such as AP-1, SP-1, GRE, SRE, etc.

Physiological effects of Aptamers. The present inventors developed thioaptamers that enhance the innate immune response by targeting the Toll-like receptor (TLR) family in mammals, which is a family of transmembrane proteins characterized by multiple copies of leucine rich repeats in the extracellular domain and IL-1 receptor motif in the cytoplasmic domain (Akira et al., 2001; Medzhitov, 2001). The TLR family is a phylogenetically conserved mediator of innate immunity that is essential for microbial recognition. Ten human homologs of TLRs (TLR1-10) have been described. By using a BLAST search, Hemmi, et al., 2000, have identified and subsequently isolated a cDNA coding for TLR9. Gene knockout experiments suggest that TLR9 acts as a receptor for unmethylated CpG dinucleotides in the bacterial DNA. Human and mouse TLR9 share an overall amino-acid identity of 75.5%. TLR9 is highly expressed in spleen (Krieg, 2002).

The immunostimulatory properties of bacterial DNA appears to be related to short six base sequences called CpG motifs that have the general structure of two 5′ purines, an unmethylated CpG motif, and two 3′ pyrimidines (Krieg, 2002). Though such sequences rarely appear in mammalian DNA due to CpG suppression and methylation of cytosine nucleotides, they are relatively abundant in bacterial DNA, occurring at the expected frequency (1 in 16) and in unmethylated form. Indeed, studies have found ODNs containing these sequence motifs to be strongly immunostimulatory, resulting in the activation of B cells, NK cells, and antigen-presenting cells, and in the induction of a variety of cytokines including interleukin-12 (IL-12), IL-6, and tumor necrosis factor-α. CpG ODNs have also been found to be effective as adjuvants in inducing antigen-specific T-helper-1-like responses, and have been the focus of much interest for their inclusion in anti-tumor vaccines and use in other therapeutic applications (Klinman et al., 1999; Krieg et al., 1999). Adjuvants enhance nonspecifically the immune response to an antigen. For example, pathogenic Arenaviruses appear to block or modify immunoregulatory cell signaling pathways (Peters & Zaki, 2002, Solomon and Vaughn, 2002; Fennewald et al., 2002). Using the present invention it was possible to disrupt Arenavirus and Flavivirus cell signals that contribute to immune evasion and pathogenesis. Using thioaptamers it was demonstrated that the thio-modified aptamers of the present invention could be used to counteract viral induced cellular perturbations and protect the infected host.

Biochemical and physiological effects of thioaptamers during viral infection—viral strategies to manage the host. During the co-evolution of viruses and their hosts, viruses have developed ingenious strategies to counteract the host defenses that normally control viral replication and spread. Similarly, viral strategies modify the cellular environment to promote viral macromolecular synthesis and viral replication. This highly ordered interaction often has the unfortunate consequence of inducing disease in the host. Viruses have evolved mechanisms to interfere with major histocompatibility complex antigen presentation, block apoptosis, disrupt complement cascades and modulate multiple cytokine networks (Lalani & McFadden, 1999; Ploegh, 1998). Viruses have targeted cell-signaling pathways involved in cytokine and chemokine signaling, the regulation of apoptosis, and the cell cycle. Studies have revealed a number of instances of direct viral intervention in the receptor and receptor proximal signaling, as well as direct interaction with signaling kinase cascades and transcription factors (McFadden et al., 1998; Ploegh, 1998; Hiscott, 2001; Hiscott et al., 2001). Most examples have come from large DNA viruses with sufficient coding capacity to encode viral homologs of cellular proteins. These viruses use molecular mimicry to exploit the cellular environment to promote viral replication and antagonize the immune response to sustain their survival in an immunocompetent host (Cameron et al., 1999; Willer et al., 1999; Hiscott et al., 2001). Influencing key transcription factors that regulate pro or anti-inflammatory cytokines is an efficient means by which viruses could cripple multiple immune responses (Powell et al., 1996; Tait et al., 2000). The strategies employed by the smaller, less genetically complex viruses are equally elegant, and often even more of an enigma.

Pichinde infection of guinea pigs is particularly suited to studies on the immunomodulation by virus infection. There are two virus variants with minimal genomic differences but profoundly different effects on the animal. Infection by the P2 variant of virus results in mild illness from which the animal recovers. Infection by the P18 variant results in death. These two virus variants were used to distinguish an effective immune response against the P2 virus, from an ineffective response against the P18 virus.

Using the thioaptamers of the present invention, the differential effect of virus infection was identified as including a profound effect on the transcription factors NF-κB and RBP-Jκ. Data generated by the present inventors (Fennewald et al., 2002) showed differential alterations in the transcription factors NF-κB and RBP-Jκ in P2 and P18 virus-infected guinea pig peritoneal macrophages. The P2 variant shows less NF-κB present and a higher mobility RBP-Jκ complex. This observation was used in an animal model of arenavirus disease in which two virus variants differentially affect target cell signaling pathways. NF-κB and AP-1 (CREB) family members are key regulators of the immune response and transcription factors involved interferon response to virus infection all are differentially induced in pathogenic Pichinde infections. Using the thioaptamers of the present invention infected hosts virulence was reduced by modulating virus induced alterations in cellular signal transduction.

The effect of contacting a cell with a thioaptamer and the measurement of a modification in a biological activity was determined both in vitro and in vivo. Many of the signaling pathways and transcription factors activated during immune system activation lead to the synthesis of the inflammatory cytokines. Certain pathways require the expression of various cytokines. The effect of the virus variants (and polyI/C) on the induction of cytokines was determined in vitro.

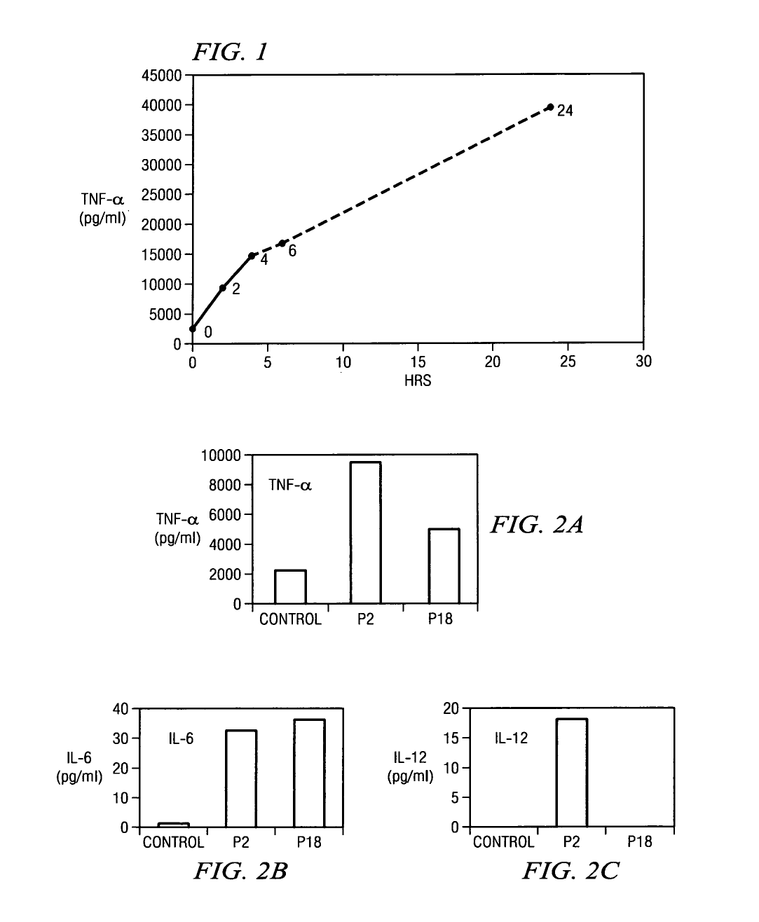

FIG. 1 is a graph that shows that polyI/C is an effective inducer of the proinflammatory cytokine TNF-α. Infection with Pichinde P2 (P2) and Pichinde P18 (P18) also alter the expression of this and other inflammatory cytokines. In particular, P2 and P18 were able to induce equally cytokines such as IL-6; which are moderately different in their induction of TNF-α and substantially different in IL-12 induction (compares FIGS. 2A, 2B, 2C). Thus, differences in signaling and inflammatory responses are associated with immune activation by P2 virus and poor activation by the P18 virus. For example, IL-12 is especially important in directing the anti-viral immune response to the effective Th1 cytotoxic T cell response (Seow, 1998). In addition to supporting the association with the immune response, this data can be used to direct the transcription factors to target. For example, IL-6 induction is similar for both virus variants.

To target transcription factors key in regulating TNFα and IL12 and other key mediators of the immune response two thioaptamers were produced, XBY-6 (SEQ ID NO.: 1) targeting NF-κB p50 homodimers and XBY-S2 targeting AP-1, both with six dithio residues. In FIG. 4, XBY-S2 (SEQ ID NO.: 2) is demonstrated to bind specifically to AP-1 proteins in pre-B cell nuclear extracts (70Z/3) and to human recombinant c-jun protein dimers (AP-1). Supershift analyses indicate that XBY-S2 binds to several members of the AP-1 protein family including JunD, CREB and possibly ATF2, and c-Jun. The XBY-6 thioaptamer binds specifically to the NF-κB p50 (or p105) homodimer (data not shown). Macrophage cultures were treated with XBY-S2 and XBY-6 and nuclear extracts were produced to assay the effects of these thioaptamers on the DNA binding activities of the transcription factors to which they are targeted. Macrophage cultures were treated with liposomes, and liposome containing the indicated thioaptamers overnight and nuclear extracts produced and assayed using the indicated oligonucleotides. The XBY-S2 thioaptamer efficiently eliminated transcription factor binding to the AP-1 oligonucleotide. In contrast, treatment with XBY-6 resulted in an increase in the NF-κB DNA binding activity.

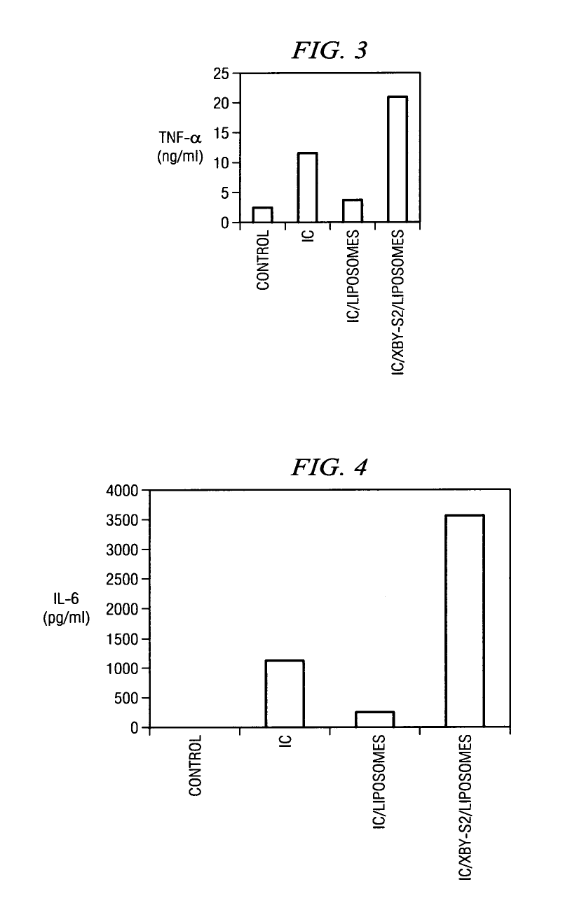

In order to determine the consequence of the elimination of AP-1 DNA binding activity by XBY-S2, separate stimulated macrophage cultures were incubated with XBY-S2 and PolyI/C and TNFα and IL-6 release measured from the culture media. The expression of both TNFα and IL-6 are increased in response to polyI/C (FIGS. 3 and 4). Pretreatment of cultures with XBY-S2 thioaptamer increases the amount of both cytokines produced in response to poly I/C. These results indicate that elimination of AP-1 from cells by the XBY-S2 decoy thioaptamer increases the production of cytokines.

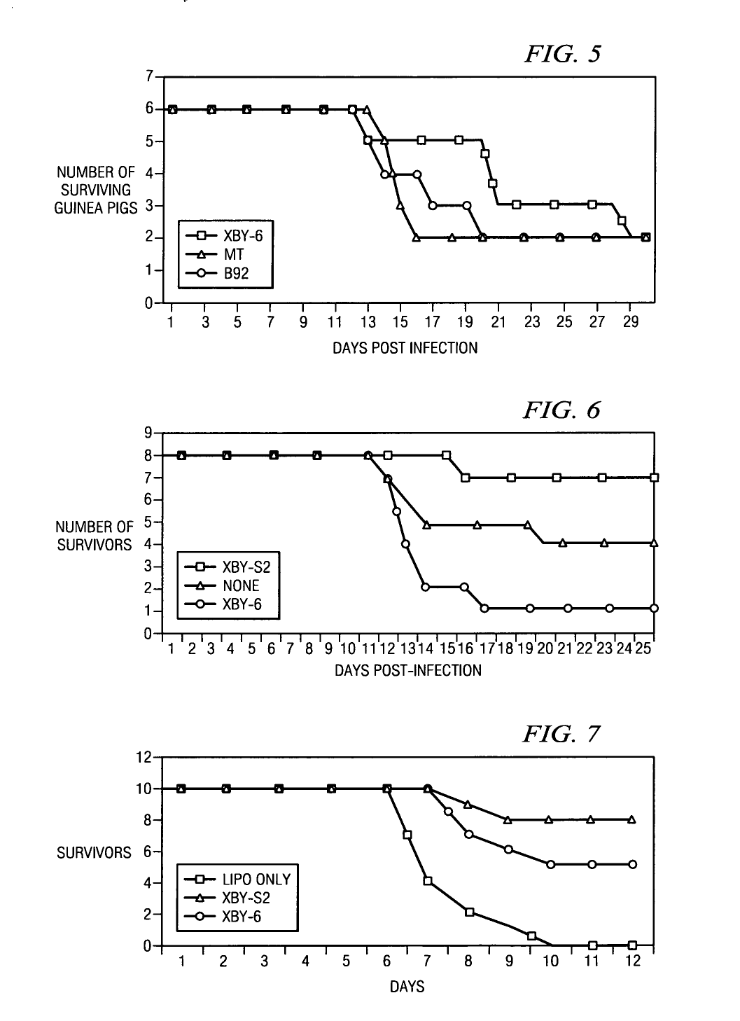

Next, the effect on a detectable biochemical activity in vivo using a thioaptamer was determined. It has been suggested that arenavirus and West Nile virus pathogenesis is the result of viral perturbation of the immune response resulting in the inappropriate expression of cytokines. Therefore, the modulation of cell signaling by appropriate thioaptamers that may reverse the inappropriate gene expression and help to alleviate the symptoms and perhaps prevent host death was determined. Guinea pigs were treated with the XBY-6 thioaptamer targeting NF-κB p50 homodimers at days 0, 1, and 2 day relative to time of infection with a lethal dose of Pichinde virus. FIG. 5 demonstrates that the thioaptamer prolongs the survival of Arenavirus infected animals. A thioaptamer of the same base content but scrambled in sequence and containing CpG islands did not prolong survival (B92; FIG. 5). Using the XBY-S2 thioaptamer, 50-80% protection of mice from a lethal West Nile virus infection was demonstrated (Tables 1 and 2) as well as prolongation of Pichinde virus survival similar to XBY-6 (data not shown).

TABLE 1
Female 3-4 week-old NIH Swiss mice were given aptamers at one day
before and 90 minutes before administration of 10 LD₅₀ WN virus
strain USA99b by the ip route.

| Group | # surviving [%] | AST (days ± SD) |
|---|---|---|
| PBS only | 0/5 [0] | 7.2 ± 0.4 |
| Liposomes only | 0/5 [0] | 8.0 ± 0.7 |
| XBY-S2 | 4/5 [80] | 9 |
| XBY-6 | 4/5 [80] | 11 |

Based on the preliminary results obtained with XBY-6 thioaptamer and Pichinde virus, it was determined if XBY-6 or XBY-S2 would have any antiviral activity against flaviviruses. West Nile virus was selected as a model system due to its high virulence in the mouse model. Mice were challenged with a low dose of virus (i.e., 30 pfu≈10 LD₅₀). The thioaptamers (10 μg) were delivered IP in T fx50 liposomes and administered in two doses (one day before and 90 minutes before virus challenge). Control mice given PBS or liposomes succumbed to WN virus infection, while 80% of thioaptamer XBY-S2 treated animals survived challenge and remained healthy (Table 5). It was noted that both thioaptamers had antiviral activity. These results suggest that the mechanism of protection may involve binding of XBY-6 to NF-κB or XBY-S2 to AP-1, however, the present invention is not limited to, or restricted by, any one mechanism and/or theory of operation.

In previous studies with West Nile virus the present inventors had observed that animals had a brief viremia that peaked on day 3 post infection, prior to viral brain invasion. As such, three animals from each test group were sacrificed on days 3 and 6 post infection to determine viremias and virus infectivity levels in the brain. Accordingly, the protocol from the first study was repeated with increased group sizes of 16 mice (of which 6 would be sampled) and increasing the virus challenge to 100 LD₅₀ virus. As shown in Table 2, both control groups (PBS and liposomes) succumbed to challenge with WN virus while the thioaptamer-treated mice survived and remained healthy. The proportion of mice treated with XBY-S2 thioaptamer who survived challenge was the same in both studies (80%) while XBY-6 treatment protected 50% of mice in the second study as compared to 80% of mice in the first study. These differences were not statistically significant given the small sample sizes.

To obtain fundamental information on the mechanism of protection, viremias and brain infectivity titers were measured in three mice sampled from each group on days 3 and 6 post infection (Table 3). As expected, in the control groups (PBS and liposome) viremia was detected on day 3 prior to invasion of the brain; virus was detectable in the brains on day 6 post infection. The thioaptamer treated mice had reduced or undetectable viremias on day 3 post infection and no detectable virus infectivity in brains on day 6 post infection. These data indicate that the thioaptamer causes a reduction in the extraneuronal replication of the virus (as seen in the reduced viremias) and that there is insufficient virus to invade the central nervous system and cause encephalitic disease. The difference between virulent neuroinvasive strains of WN virus and poorly neuroinvasive attenuated WN strains may be explained by these results. Two mechanisms seem possible, although the invention is in no way limited by hypothesis: 1) first, the thioaptamer induces an immune response against WN virus; or 2) the thioaptamer blocks the WN virus replication. The thioaptamer may be inducing localized interferon (or other mediators of the innate immune response) that inhibits replication of the virus since the thioaptamer includes double-stranded DNA while double-stranded RNA is known to be an efficient inducer of interferon.

TABLE 2
Study 2: Female 3-4 week-old NIH Swiss mice were given aptamers at
one day before and 90 minutes before administration of 100 LD₅₀ WN
virus strain USA99b by the ip route.

| Group | # surviving [%] | AST(days ± SD) |
|---|---|---|
| PBS only | 0/10 [0] | 8.3 ± 0.8 |
| Liposomes only | 0/10 [0] | 7.7 ± 1.1 |
| XBY-S2 | 8/10 [80] | 8.5 ± 0.7 |
| XBY-6 | 5/10 [50] | 8.0 ± 0.7 |

To investigate the activity of the modified thioaptamers and the antiviral mechanism of action of the thioaptamers, the susceptibility of thioaptamer-protected mice virus to challenge was tested. Thioaptamer-treated mice from the second study who survived WN virus infection were challenged at 21 days post-infection with 100LD₅₀ of WN virus. All mice, including mock-infected controls from study 2 succumbed to virus challenge. This result indicates that there was insufficient virus replication in thioaptamer-treated mice to induce an adaptive immune response. This would suggest that the mechanism of action of the thioaptamer is either innate immunity or direct antiviral activity of the thioaptamer.

TABLE 3
Viremia and brain infectivity titers for Study 2 (see Table 6)

| | Day 3 | | Day 6 | |
|---|---|---|---|---|
| | Serum titer | Brain titer | Serum titer | Brain titer |
| Sample | (pfu/mL) | (pfu/brain) | (pfu/mL) | (pfu/brain) |
| XBY-6 #1 | 30,000 | —* | — | — |
| XBY-6 #2 | — | — | — | — |
| XBY-6 #3 | 700 | — | — | — |
| XBY-S2 #1 | 100 | — | — | — |
| XBY-S2 #2 | — | — | — | — |
| XBY-S2 #3 | — | — | — | — |
| Lipo #1 | 2,000 | — | — | 500,000 |
| Lipo #2 | 2,500 | — | — | 6,500,000 |
| Lipo #3 | 15,000 | — | — | 3,500 |
| PBS #1 | 25,000 | — | 100 | 5,500,000 |
| PBS #2 | 20,000 | — | — | 180,000,000 |
| PBS #3 | 4,500 | — | — | 2,500,000 |

*— indicates no virus detected; limits of detection were 50 pfu/ml of serum and 25 pfu/brain

Whether thioaptamers exhibited direct antiviral activity in cell culture was also determined. The direct antiviral activity of the thioaptamer was investigated in cell culture. Using six-well dishes containing Vero cells, duplicate wells were treated with one of the following samples:
1. Liposomes+xbyc2 (10 μg/well)
2. Liposomes+xbys1 (10 μg/well)
3. Liposomes+XBY-S2 (5 μg/well)
4. Liposomes+XBY-S2 (10 μg/well)
4. Liposomes only
5. Buffer only

Wells were incubated for 12 hours with the samples above and then challenged with WN virus at a multiplicity of infection (MOI) of 0.1. Samples were harvested from each well at 0, 14, 24, 34 and 48 hours. No cytopathic effect was seen until 48 hours post virus infection. Each well was assayed at each time point by hemaggluttination (HA) assay to detect the presence of virus particles. All samples showed no detectable HA (i.e., ≦4 HAU) except for the samples at 48 hours post virus infection when all wells had 32-64 HAUs. These results demonstrate that the thioaptamers have no direct antiviral activity.

One potential explanation for the antiviral activity of thioaptamers is induction of interferon. This hypothesis was investigated by taking groups of four 34 week-old female NIH Swiss and treat them with either 10 μg of XBY-S2 in liposomes, liposomes only, or buffer only on day 0 and day 1 post infection, followed by sacrificing mice on day 2 post infection. Serum samples were diluted 1 in 3 and run in ELISAs to detect mouse interferon-α/β, interferon-γ, or TNF-α. None of these cytokines was detected in the serum of any of the 12 mice sampled suggesting that interferon was not involved in the antiviral activity induced by thioaptamer XBY-S2.

FIG. 5 and Tables 1, 2 and 3 demonstrate that the survival of P18 virus infected animals can be prolonged using thioaptamers and thioaptamers can protect the majority of the animals infected with West Nile virus. These results demonstrate that modified thioaptamers alter the outcome of in vivo viral infections by Category A and B agents by the manipulation of transcription factors involved in the immune response.

FIG. 6 is a graph that shows survival curves following Pichinde P18 infection in guinea pigs treated with the NF-κB aptamer, XBY-6, the scrambled control, B92, or vehicle, MT, of animals infected by injection of 1000 pfu of Pichinde P18 at day 0, treatment consisted of intraperitoneal injections at days 0, 1 and 2;

FIG. 7 is a graph that shows survival curves of guinea pigs with thioaptamers for infection by arenavirus. The graph in FIG. 7 shows survival curves following West Nile Virus infection in guinea pigs treated with the NF-κB aptamer XBY-6, the AP-1 aptamer XBY-S2, or the liposome vehicle of animals infected by injection with lethal doses of West Nile Virus. Therefore, the thioaptamer may be used to affect a biological activity that was measured using animal survival.

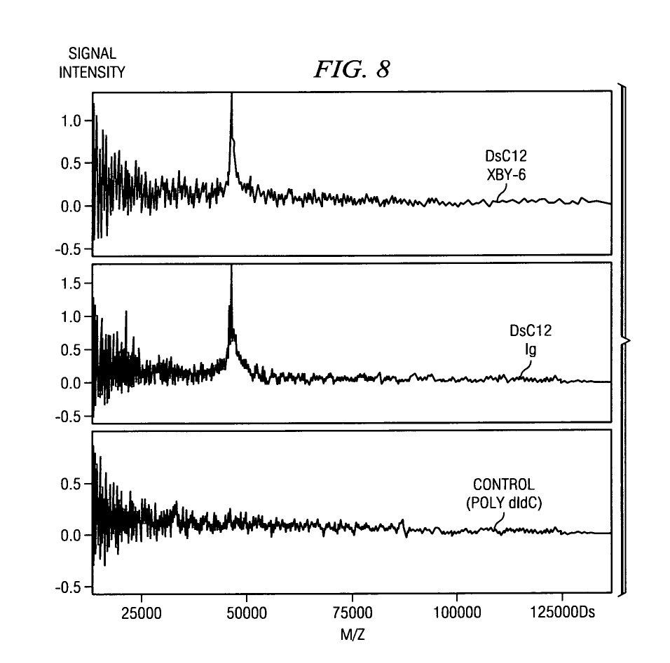

Measurement of physiological response to thioaptamer exposure by SELDI MS detection of NF-κB bound to thioaptamer surfaces and beads. The present inventors have demonstrated that thioaptamers bind both purified, recombinant NF-κB p50 and nuclear extracts on either beads (or Ciphergen PBSII ProteinChip surfaces). FIG. 8 shows three spectra using SELDI MS of p50 binding to various ProteinChips and beads. In FIG. 8 (top plot), SELDI mass spectrometry was used to detect recombinant p50 with using epoxy-activated ProteinChip Arrays. Duplex aptamers with a 5′-amino terminus linked to a 12 carbon chain were synthesized. These duplex thioaptamers were the dithioate 14-mers XBY-6 (C12-XBY-6, top), the normal phosphate backbone 22-mer NF-κB binding site with the C12 5′-amino linker (C12-IgκB, middle) or a non-specific, non-covalently linked duplex (polydIdC, bottom) as a control. These aptamers were spotted individually onto spots of a preactivated ProteinChip Array (PS20) in 2 μl of 25 mM NaHCO₃ (pH 9) and incubated overnight at room temperature and high humidity. Following incubation, excess thioaptamer was removed by washing 2 times in 5 μl 25 mM PBS, 0.1% Triton X-100 (pH 7.2) and the surface was blocked to limit non-specific binding with 1 μl of 100 μM bovine serum albumin for 4 hrs. After blocking, excess BSA was washed away as above. Next, 4.3 pmol recombinant p50 was spiked into 100 pmol BSA in 5 μl of optimized EMSA buffer containing 20 mM DTT, 0.01 μM polydIdc and incubated on each of the aptamer/thioaptamer surfaces for 2 hrs at room temperature and high humidity. Following incubation, each spot was washed with 5 μl of 50 mM Tris buffer (pH 7.2), 0.1% CHAPS, 1 M urea, 0.5 M NaCl, followed by a water wash to remove all non-specific binding components. 0.8 μl Sinapinic acid (saturated solution in 50% acetonitrile, 0.5% trifluoroacetic acid) was added to each spot, dried and the array analyzed in the mass reader. As shown in FIG. 8, the p50 (MW˜46,200) on either the XBY-6 or IgκB bound surfaces was detected, but not the control. In other spectra with more stringent washing, the XBY-6 spot, but not the IgκB spot, was shown to retain the bound p50 (spectra not shown), confirming the tighter binding of p50 to XBY-6 (sub-nM) relative to IgκB (K_{D} 4 nM).

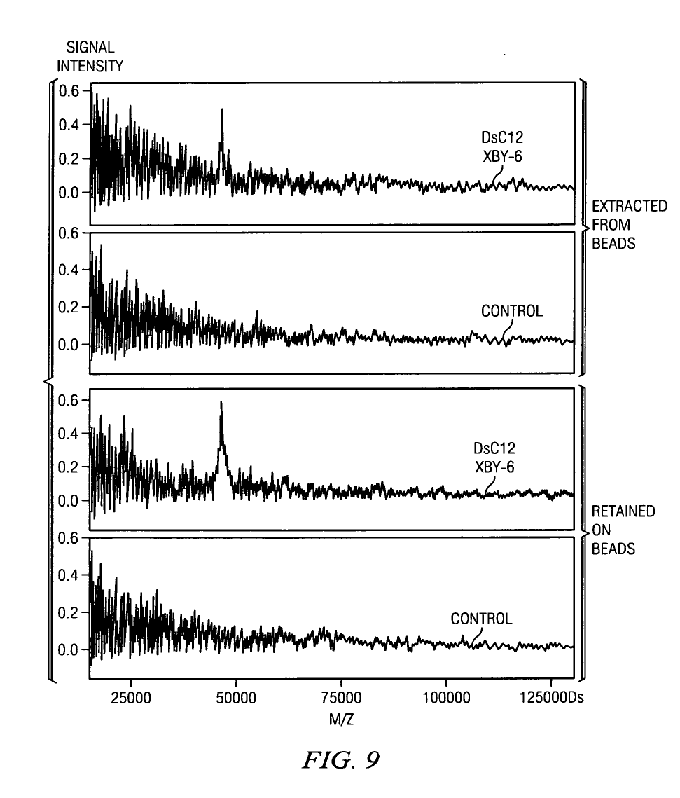

The panels in FIG. 9 show that the XBY-6 thioaptamer can also capture recombinant p50 (MW˜46,200) on gel beads to which the 5′amino-C12 linked XBY-6 is coupled to 20 ul (1:1) AminoLink® Plus Coupling gel (Pierce, Immunoprecipitation kit, cat # 45335). In this study, 3 μg of C12-XBY-6 was coupled overnight at 4° C. following the kit protocol. After quenching the gel, 6 μg of p50 in 1X EMSA buffer with polydIdC was added to the gel and incubated for 2 hrs with shaking at room temperature. The gel was washed to remove nonspecifically bound proteins, followed by one quick rinse with water. Protein bound to the gel was extracted with 5 μl of organic solvent (50% AcN and 0.01% TFA) with shaking for 20 min. All of the extracts were spotted onto NP20 ProteinChips, dried, followed by addition of saturated SPA and read on the Ciphergen PBSII MS (top two spectra). After extraction, 1 μl of the gel was loaded onto NP20 chip (bottom two spectra). Proteins still bound to the gel was analyzed using saturated SPA on the PBSII. Once again it was found that p50 can be identified by SELDI, both in the extract and retained directly on the beads.

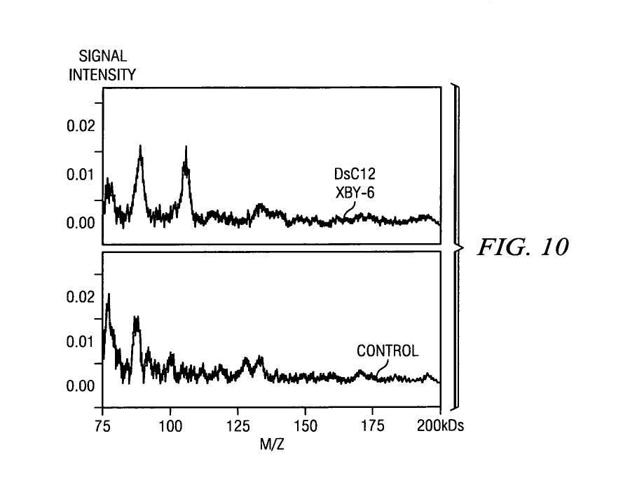

FIG. 10 shows the capture of nuclear extracts onto Ciphergen's PS20 ProteinChip Arrays: Either 0.5 μg of C12-XBY-6 (top) or 0.5 μg of poly dIdC (bottom) were incubated on PS20 chip overnight. The chips were blocked with 7 mg/ml BSA in PBS/0.1% Tween-20. Following blocking, 49 μg of nuclear extract in optimized EMSA buffer were incubated on each spot for 2 hr with shaking. Each spot was washed with PBS/0.1% Triton three times, followed by one quick wash with water. Proteins bound on each spot were analyzed using saturated SPA on the PBSII. These results indicate that a protein was bound with a MW ˜105,591, which may represent p105, the precursor to p50 or the p50/p50 homodimer.

Bead-based phosphorodithioate and phosphorothioate thioaptamer combinatorial libraries and high throughput sorting against targeted proteins. The one-bead, one-aptamer split synthesis method disclosed herein was used to identify a specific ODN aptamer that targets proteins or other biomolecules. In combination with the split and pool synthesis combinatorial chemistry method for creating a combinatorial library of oligonucleotide agents (either phosphate, monothiophosphate or dithiophosphate; Gorenstein, et al., U.S. Pat. No. 6,423,493, relevant portions incorporated herein by reference) both monothiophosphate and dithiophosphate combinatorial libraries attached to individual support beads were shown to produce aptamers that demonstrate target-specific binding. Proteins that bind tightly to only a few of the 10⁴-10⁸ different support beads may be selected by binding either purified proteins, nuclear or cytoplasmic extracts or pools of proteins to the beads and then identifying which beads have bound target protein by immunostaining, fluorescent staining techniques or MS (SELDI). Thus, the methods and compositions created and isolated thereby allow for rapid screening, isolation and identification of specific thioaptamers that bind to proteins such as NF-κB and AP-1 using the PCR-based identification tag of the selected bead disclosed herein.

Aptamers target and eliminate NF-κB protein dimers that function as repressors of transcription. Although most of the NF-κB proteins are transcriptionally active, some combinations are thought to act as inactive or repressive complexes. It is well-known that the p50/RelA(p65), RelA(p65)/RelA(p65) and RelA(p65)/c-Rel are all transcriptionally active, whereas the p50 or p52 homodimers are transcriptionally repressive. This is due to the lack of a variable C-terminal domain in p50 and p52 that is found in the activating NF-κB/Rel proteins. The C-terminal domain is most likely responsible for the transactivation of NF-κB-responsive genes. Therefore, aptamers targeting p50 or p52 homodimers would inactivate naturally occurring NF-κB dimers that normally repress transcription. As such, the inactivation of these transcriptional repressors would increase the expression of genes to whose promoters they would normally be bound. NF-κB proteins play a critical role in innate immunity as NF-κB proteins play critical roles in the activation of immune cells by upregulating the expression of many cytokines essential to the immune response. In particular, NF-κB stimulates the production of IL-1, IL-6, TNFα and NF-κB themselves in an autoregulatory feedback loop. All of these cytokines and other molecules have multiple effects that contribute to inflammation. Therefore, using the present invention an increase in the expression of proinflammatory cytokines and other immunomodulatory proteins in immune cells treated with aptamers that target p50 or p52 homodimers would result in an enhanced level of innate immunity. However, the present invention may be used to either repress or enhance the innate immune response as these transcription factors can serve as both repressors and activators of transcription. For example, p50 homodimers have been shown to activate transcription when associated with Bcl3. Hence, in cells containing Bcl3, the same aptamer will prevent p50 transcriptional activation. Families of transcription factor often include some that serve as either transcriptional activators or repressors. Therefore the present invention can be used more generally to target other transcription factor targets.

Temporal NF-κB responses and aptamer intervention. Biochemical and genetic analyses have established NF-κB as an inducible transcription factor wherein the majority is maintained as an inactive complex through the cytoplasmic retention by a family of IκB inhibitory proteins. Stimulation of cells by a variety of signals leads to the proteolytic degradation of IκB proteins and subsequent nuclear translocation of NF-κB. Depending on the inducer and/or the cell type, a variety of activation profiles of NF-κB have been described. These activation profiles can differ in the kinetics of activation. Furthermore, the NF-κB proteins involved in a given response can change at different times in this continuum of the NF-κB response to a given stimulus. The resynthesis of IκB proteins is in part responsible for which NF-κB proteins remain nuclear and active. Whereas NF-κB is generally regarded as a key player in the initiation of the immune response, innate and adaptive immunity, more recent information suggests the NF-κB is also implicated in the resolution of inflammation.

For example, late phase activation of NF-κB is associated with the expression of cyclooxygenase 2-derived anti-inflammatory prostaglandins and the anti-inflammatory cytokine, transforming growth factor-β1. Inhibition of NF-κB during the resolution of inflammation protracts the inflammatory response and prevents apoptosis. Therefore, complete suppression of the NF-κB pathway or suppression of certain NF-κB dimers at inappropriate times during inflammation is likely to be contraindicated. Since the NF-κB response is dynamic in nature, so must be treatments using aptamers where different NF-κB dimers are targeted at various times in the disease process. The different thioaptamers disclosed herein may be used in treatment of viral infections in a timed sequence. The initial treatment would involve using a thioaptamer binding a transcription factor whose inactivation would result in a boost to the immune system. At later times following viral challenge, the treatment would use a thioaptamer which binds a transcription factor so as to suppress cytokine-induced shock.

Thioaptamer based chips may be used in conjunction with therapy to monitor the host transcription factor responses to viral challenge and determine the appropriate thioaptamer to be used in treatment. Transcription factor families can have some redundancy in the functions of some of their members. Further, transcription from genes is generally regulated by the simultaneous binding and actions of multiple transcription factors. These facts would suggest that treatment could also require the use of varying combinations of thioaptamers targeting multiple transcription factors at different times before and after infection.

The present inventors have synthesized successfully a monothio RNA library (2¹⁵=32768) to target and eliminate NF-κB protein dimers that function as repressors of transcription (Gorenstein, et al., patent pending, 2002). Thus, standard phosphoramidite (DNA and RNA) chemistry was used for the thioaptamer RNA library. A 0.5 M 1H-tetrazole in acetonitrile was used as DNA activator. A 0.5 M solution of DCI (dicyanoimidazole) in acetonitrile was used as RNA activator. The libraries were prepared on a 1 μmole scale of polystyrene beads (66-70 μm). The downstream and upstream primers, 5′-d(GGATCCGGTGGTCTG)-3′ and 5′-d(CCTACTCGCGAATTC)-3′ were synthesized in parallel on a two-column DNA synthesizer (Expedite 8909, Applied Biosystems). Following the 5′-primer, the sequences programmed on the synthesizer for the combinatorial mono RNA library were 5′-r(GA*UC*CU*GA*AA*CU*GU*UU*UA*AG*GU*UG*GC*CG*AU*C)-3′ (SEQ ID NO.: 24) on column 1 and 5′-r(cU*aG*gA*cU*uG*gC*aC*aA*cC*gU*cA*cA*cU*gC*uA*u)-3′ (SEQ ID NO.: 25) on column 2. The 3′-primer sequence completed the 61-mer programmed on the synthesizer. A “split and pool” occurred at each position indicated by an asterisk in order to synthesize the combinatorial region for the monothio RNA. The lower case letter indicates a 3′-thioate linkage, the upper case letter indicates a 3′-phosphate linkage. The coupling yield was typically upwards of 98.5% as determined by the dimethoxytrityl cation assay (DNA couplings are typically >99%/nt). Sulfurization chemistry utilized the Beaucage reagent. The fully protected monothio RNA combinatorial library with the non-cleavable linker beads were treated with 4 ml of a mixture of 3:1 (v/v) (28%) NH3: EtOH at 39° C. for 21 hrs. The beads were centrifuged, the supernatant was removed and the solid support was washed with double-distilled water. After lyophilization the solid support was treated with 2 ml of triethylamine trihydrofluoride (TEA-3HF) for 20 hrs at room temperature. Again, the beads were centrifuged, the supernatant was removed and the solid support was washed with double-distilled water. RT PCR and TA cloning confirmed the successful synthesis of the ssRNA thioaptamer library.

TABLE 3. Sequences of thioaptamers selected from split synthesis (small letters indicate thiophosphate 3′ to base).

| | |
|---|---|
| 5′-tGTGcAGGGACTgAtGaCGGt-3′ | (SEQ ID NO.: 26) |
| 5′-CtGTGCatCGAaGTTtGCAtTt-3′ | (SEQ ID NO.: 27) |
| 5′-AtGcAcAtCtCaGgAtGaCGGt-3′ | (SEQ ID NO.: 28) |
| 5′-AGTTGcAGGtCaGgACCCAtTt-3′ | (SEQ ID NO.: 30) |

Flow cytometry sorting of thioaptamer bead-based library. The present inventors have also demonstrated the successful application of high throughput/multi-color flow cytometry and bead sorting to screen aptamer bead libraries for those beads which bind to, e.g., a target protein (Gorenstein, et al., patent pending, 2002). Modifications were made to a custom-built flow cytometer to make it more amenable to bead identification and isolation. For example, bead fluorescence and forward scatter were the two parameters chosen for real-time characterization of each aptamer bead passing the first sort point of the custom-built flow cytometer/sorter. Other scanning and sorting parameters may be used to select, isolate, view, designate, characterize, etc. the beads through a flow cytometer.

In operation, “positive” beads (contain thioaptamer-bound target protein, the target protein was fluorescent-labelled with Alexa 488 dye) were easily sorted from negative beads. Flow cytometry may be used to replace, e.g., visual fluorescence microscope identification of beads containing bound target protein and the need to isolate the individual “positive” beads with the micromanipulator described previously. The flow-sorted “positive” beads can then be subjected to, e.g., one-bead PCR to identify the thioaptamer that binds the target protein.

TABLE 4
Population Statistics for bead sorting, WinList analyses
(all data were color-compensated)

| Sample | Total | Region | % Gate |
|---|---|---|---|
| CONTROL.FCS | | | |
| R1: Autofluorescent Beads | 10000 | 9530 | 95.3 |
| FCS | | | |
| R2: p50 Alexa 488 Positive Beads | 10000 | 35 | 0.35 |
| FCS | | | |
| R3: p65 PE Positive Beads | 20000 | 3488 | 17.44 |
| FCS | | | |
| R1: Autofl. Beads & Carrier Beads | 1000000 | 963321 | 96.33 |
| R2: p50 Alexa 488 Positive Beads | 1000000 | 354 | 0.04 |
| R3: p65 PE Positive Beads | 1000000 | 935 | 0.09 |

Fluorescence sorting was also used to demonstrate the use of the one-bead, one-ODN:protein system using dual color sorting. The IgκB dsDNA consensus sequences were immobilized onto 15-20 micron polystyrene microspheres. The DNA bound beads were then incubated with purified p50 and p65 proteins, respectively. DNA transcription factor complexes were detected with primary antibodies specific for the p50 and p65 proteins followed by an additional incubation with Alexa 488-conjugated secondary antibody for p50 and PE-conjugated secondary antibody for p65. The beads were viewed by fluorescent microscopy and then analyzed on the MCU's HiReCS system. A Control Fluorescent Cell Sort (CONTROL.FCS) shows the autofluorescent microspheres in the negative control sample where the beads were unbound. The majority of the beads in the “debris” population were the 0.8 micron carrier beads that were used to bring up the volume of the samples since the beads were at a very low dilution.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A method of disrupting selectively a target molecule within a physiological pathway comprising the steps of:
characterizing a detectable activity of the physiological pathway;
identifying a thioaptamer that modifies the activity of the target molecule of the physiological pathway; and
determining the extent of the modification to the activity of the physiological pathway in the presence of the thioaptamer.

2. The method of claim 1, wherein between one to about eight of the phosphate links of the thioaptamer are thio-modified.

3. The method of claim 1, wherein between one to about eight of the phosphate links of both strands of the thioaptamer are thio-modified.

4. The method of claim 1, wherein the thioaptamer is single stranded.

5. The method of claim 1, wherein the thioaptamer comprises RNA.

6. The method of claim 1, wherein the thioaptamer comprises siRNA.

7. The method of claim 1, wherein the target molecule is selected from the group comprising a nucleic acid, a protein, a complex of proteins and a complex of one or more proteins and one or more nucleic acids.

8. The method of claim 1, wherein the detectable activity measured comprises a chemical assay, a biological assay, a photometric, a spectrophotometric, a colorimetric, a chemiluminescent, a fluorescent, a radioactive, a mass spectrometric, a electrophoretic, magnetic resonance imaging, nuclear magnetic resonance and combinations thereof.

9. The method of claim 1, wherein no more than three adjacent phosphate sites of the thioaptamer are replaced with phosphorothioate groups.

10. The method of claim 1, wherein at least a portion of non-adjacent dA, dC, dG, or dT phosphate sites of the thioaptamer are replaced with phosphorothioate groups.

11. The method of claim 1, wherein all of the non-adjacent dA, dC, dG, and dT phosphate sites of the thioaptamer are replaced with phosphorodithioate groups.

12. The method of claim 1, wherein the thioaptamer is between about 12 and about 60 bases in length.

13. The method of claim 1, wherein substantially all non-adjacent phosphate sites of the thioaptamer are replaced with one or more phosphorothioate groups.

14. The method of claim 1, wherein the thioaptamer is defined further as being double-stranded.

15. The method of claim 1, wherein one or more modified nucleotides of the thioaptamer are selected from the group consisting of dATP(αS), dTTP(αS), dCTP(αS), dGTP(αS), rUTP (αS), rATP(αS), rCTP(αS), rGTP(αS), d ATP(αS₂), dTTP(αS₂), dCTP(αS₂), dGTP(αS₂), rATP(αS₂), rCTP(αS₂), rGTP(αS₂) and rUTP(αS₂) or modifications thereof.

16. The method of claim 1, wherein the thioaptamer is selected by the steps of:
synthesizing a random phosphodiester oligonucleotide combinatorial library using a mix of four nucleotides, wherein at least a portion of at least one of the nucleotides in the mix is thiophosphate-modified, to form a thioaptamer combinatorial library;
contacting the thioaptamer combinatorial library with the target molecule isolating a subset of thioaptamers binding to the target molecule.

17. The method of claim 16, wherein the subset of thioaptamers is sequenced.

18. The method of claim 1, wherein the physiological pathway is selected from the group consisting of biosynthesis of cofactors prosthetic groups and carriers (lipoate synthesis, riboflavin synthesis pyridine nucleotide synthesis); the biosynthesis of the cell envelopes (membranes, lipoproteins, porins, surface polysaccharides, lipopolysaccharides, antigens and surface structures); cellular processes including cell division, chaperones, detoxification, protein secretion, central intermediary metabolism (energy production of phosphorus compounds); energy metabolism including aerobic, anaerobic, ATP proton motive force interconversions, electron transport, glycolysis triose phosphate pathway, pyruvate dehydrogenase, sugar metabolism; purine, pyrimidine nucleotide synthesis, 2′-deoxyribonucleotide synthesis, nucleotide and nucleoside interconversion, salvage of nucleoside and nucleotides, sugar-nucleotide biosynthesis and conversion; regulatory functions including transcriptional and translational controls, DNA replication including degradation of DNA, DNA replication, restriction modification, recombination and repair; transcription including degradation of DNA, DNA-dependent RNA polymerase and transcription factors; RNA processing; translation including amino acyl tRNA synthetases, degradation of peptides and glycopeptides, protein modification, ribosome synthesis and modification, tRNA modification; translation factors transport and binding proteins including amino acid, peptide, amine carbohydrate, organic alcohol, organic acid and cation transport; and other systems for the adaptation, specific function or survival of an artificial organism and combinations thereof.

19. The method of claim 1, wherein the physiological pathway comprises cytokine release.

20. The method of claim 1, wherein the target molecule comprises a nuclear regulatory factor.

21. The method of claim 1, wherein the target molecule comprises a nuclear regulatory factor selected from the group consisting of NF IL-6, NF-κB, AP-1 and combinations thereof.

22. A method of testing a chemical agent comprising the steps of:
exposing a cell having a measurable biochemical activity to a thioaptamer that modifies the activity of a target molecule;
contacting at least a portion of the cell with the chemical agent; and
measuring the effect that the chemical agent has on the measurable biochemical activity.

23. The method of claim 22, wherein between one to about eight of the phosphate links of the thioaptamer are thio-modified.

24. The method of claim 22, wherein between one to about eight of the phosphate links of both strands of the thioaptamer are thio-modified.

25. The method of claim 22, wherein the thioaptamer is single stranded.

26. The method of claim 22, wherein the thioaptamer comprises RNA.

27. The method of claim 22, wherein the thioaptamer comprises a dsRNA.

28. The method of claim 22, wherein the target molecule is selected from the group comprising a nucleic acid, a protein, a complex of proteins and a complex of one or more proteins and one or more nucleic acids.

29. The method of claim 22, wherein the measurable biochemical activity is determined by an assay that is chemical, biological, photometric, spectrophotometric, calorimetric, chemiluminescent, fluorescent, radioactive, mass spectrometric, electrophoretic, magnetic resonance imaging, nuclear magnetic resonance and combinations thereof.

30. The method of claim 22, wherein the thioaptamer has SEQ ID NOS: 1-9.

31. The method of claim 22, wherein the modified nucleotide aptamer comprises one or more phosphorothioate or phosphordithioate linkages.

32. The method of claim 22, wherein one or more modified nucleotides of the thioaptamer are selected from the group consisting of dATP(αS), dTTP(αS), dCTP(αS), dGTP(αS), rUTP (αS), rATP(αS), rCTP(αS), rGTP(αS), d ATP(αS₂), dTTP(αS₂), dCTP(αS₂), dGTP(αS₂), rATP(αS₂), rCTP(αS₂), rGTP(αS₂) and rUTP(αS₂) or modifications thereof.

33. The method of claim 22, wherein the measurable biochemical activity is selected from the group consisting of biosynthesis of cofactors prosthetic groups and carriers (lipoate synthesis, riboflavin synthesis pyridine nucleotide synthesis); the biosynthesis of the cell envelopes (membranes, lipoproteins, porins, surface polysaccharides, lipopolysaccharides, antigens and surface structures); cellular processes including cell division, chaperones, detoxification, protein secretion, central intermediary metabolism (energy production of phosphorus compounds); energy metabolism including aerobic, anaerobic, ATP proton motive force interconversions, electron transport, glycolysis triose phosphate pathway, pyruvate dehydrogenase, sugar metabolism; purine, pyrimidine nucleotide synthesis, 2′-deoxyribonucleotide synthesis, nucleotide and nucleoside interconversion, salvage of nucleoside and nucleotides, sugar-nucleotide biosynthesis and conversion; regulatory functions including transcriptional and translational controls, DNA replication including degradation of DNA, DNA replication, restriction modification, recombination and repair; transcription including degradation of DNA, DNA-dependent RNA polymerase and transcription factors; RNA processing; translation including amino acyl tRNA synthetases, degradation of peptides and glycopeptides, protein modification, ribosome synthesis and modification, tRNA modification; translation factors transport and binding proteins including amino acid, peptide, amine carbohydrate, organic alcohol, organic acid and cation transport; and other systems for the adaptation, specific function or survival of an artificial organism and combinations thereof.

34. The method of claim 22, wherein the measurable biochemical activity comprises cytokine release.

35. The method of claim 22, wherein the target molecule comprises a nuclear regulatory factor.

36. The method of claim 22, wherein the target molecule comprises a nuclear regulatory factor selected from the group consisting of NF IL-6, NF-κB, AP-1 and combinations thereof.

37. The method of claim 22, wherein the cell is from an animal knock-out, knock-in, transgenic, over-expressing transgenic, under-expressing-transgenic, conditional knockout, mutant, chimera or combinations thereof.

38. A system for testing the biological effects of a molecule comprising:
at least a portion of a cell comprising a target molecule and a measurable biochemical activity;
a thioaptamer that modifies the activity of the target molecule that is added to the cell; and
a biochemical activity detection device that records differences in the measurable biochemical activity of the cell upon exposure to the molecule.

39. The system of claim 38, wherein the measurable biochemical activity is selected from the group consisting of biosynthesis of cofactors prosthetic groups and carriers (lipoate synthesis, riboflavin synthesis pyridine nucleotide synthesis); the biosynthesis of the cell envelopes (membranes, lipoproteins, porins, surface polysaccharides, lipopolysaccharides, antigens and surface structures); cellular processes including cell division, chaperones, detoxification, protein secretion, central intermediary metabolism (energy production of phosphorus compounds); energy metabolism including aerobic, anaerobic, ATP proton motive force interconversions, electron transport, glycolysis triose phosphate pathway, pyruvate dehydrogenase, sugar metabolism; purine, pyrimidine nucleotide synthesis, 2′-deoxyribonucleotide synthesis, nucleotide and nucleoside interconversion, salvage of nucleoside and nucleotides, sugar-nucleotide biosynthesis and conversion; regulatory functions including transcriptional and translational controls, DNA replication including degradation of DNA, DNA replication, restriction modification, recombination and repair; transcription including degradation of DNA, DNA-dependent RNA polymerase and transcription factors; RNA processing; translation including amino acyl tRNA synthetases, degradation of peptides and glycopeptides, protein modification, ribosome synthesis and modification, tRNA modification; translation factors transport and binding proteins including amino acid, peptide, amine carbohydrate, organic alcohol, organic acid and cation transport; and other systems for the adaptation, specific function or survival of an artificial organism and combinations thereof.

40. The system of claim 38, wherein the measurable biochemical activity comprises cytokine release.

41. The system of claim 38, wherein the target molecule comprises a nuclear regulatory factor.

42. The system of claim 38, wherein the target molecule comprises a nuclear regulatory factor selected from the group consisting of NF IL-6, NF-κB, AP-1 and combinations thereof.

43. The system of claim 38, wherein the cell is from an animal knock-out, knock-in, transgenic, over-expressing transgenic, under-expressing-transgenic, conditional knockout, mutant, chimera or combinations thereof.

44. The system of claim 38, wherein the thioaptamer comprises RNA.

45. The system of claim 38, wherein the thioaptamer comprises siRNA.

46. The system of claim 38, wherein the target molecule is selected from the group comprising a nucleic acid, a protein, a complex of proteins and a complex of one or more proteins and one or more nucleic acids.

47. The system of claim 38, wherein the detectable activity measured comprises a chemical assay, a biological assay, a photometric, a spectrophotometric, a calorimetric, a chemiluminescent, a fluorescent, a radioactive, a mass spectrometric, a electrophoretic, magnetic resonance imaging, nuclear magnetic resonance and combinations thereof.


SEQUENCE LISTING (22 sequences)

SEQ ID NO: 1 - DNA, 14 bp, Artificial Sequence
  Description of Artificial Sequence Synthetic oligonucleotide
ccaggagattccac

SEQ ID NO: 2 - DNA, 14 bp, Artificial Sequence
  Description of Artificial Sequence Synthetic oligonucleotide
ccagtgactcagtg

SEQ ID NO: 3 - DNA, 14 bp, Artificial Sequence
  Description of Artificial Sequence Synthetic oligonucleotide
ttgcgcgcaacatg

SEQ ID NO: 4 - DNA, 14 bp, Artificial Sequence
  Description of Artificial Sequence Synthetic oligonucleotide
ccagtgactcagtg

SEQ ID NO: 5 - DNA, 14 bp, Artificial Sequence
  Description of Artificial Sequence Synthetic oligonucleotide
ttgcgcgcaacatg

SEQ ID NO: 6 - DNA, 21 bp, Artificial Sequence
  Description of Artificial Sequence Synthetic oligonucleotide
tgtgcagggactgatgacggt

SEQ ID NO: 7 - DNA, 22 bp, Artificial Sequence
  Description of Artificial Sequence Synthetic oligonucleotide
ctgtgcatcgaagtttgcattt

SEQ ID NO: 8 - DNA, 22 bp, Artificial Sequence
  Description of Artificial Sequence Synthetic oligonucleotide
atgcacatctcaggatgacggt

SEQ ID NO: 9 - DNA, 22 bp, Artificial Sequence
  Description of Artificial Sequence Synthetic oligonucleotide
agttgcaggtcaggacccattt

SEQ ID NO: 10 - DNA, 62 bp, Artificial Sequence
  Description of Artificial Sequence Synthetic oligonucleotide
atgcttccacgagcctttcnnnnnnnnnnnnnnnnnnnnnnctgcgaggcggtagtctattc

SEQ ID NO: 11 - DNA, 22 bp, Artificial Sequence
  Description of Artificial Sequence Synthetic oligonucleotide
gggcgtatatgtgtggcggggg

SEQ ID NO: 12 - DNA, 21 bp, Artificial Sequence
  Description of Artificial Sequence Consensus sequence
ggggtgntgtsssgngsnsnc

SEQ ID NO: 13 - DNA, 18 bp, Artificial Sequence
  Description of Artificial Sequence Primer
atgcctactcgcgaattc

SEQ ID NO: 14 - DNA, 19 bp, Artificial Sequence
  Description of Artificial Sequence Primer
gaacaggaccaccggatcc

SEQ ID NO: 15 - DNA, 15 bp, Artificial Sequence
  Description of Artificial Sequence Primer
ggatccggtggtctg

SEQ ID NO: 16 - DNA, 15 bp, Artificial Sequence
  Description of Artificial Sequence Primer
cctactcgcgaattc

SEQ ID NO: 17 - RNA, 31 nt, Artificial Sequence
  Description of Artificial Sequence Synthetic oligonucleotide
gauccugaaacuguuuuaagguuggccgauc

SEQ ID NO: 18 - RNA, 31 nt, Artificial Sequence
  Description of Artificial Sequence Synthetic oligonucleotide
cuaggacuuggcacaaccgucacacugcuau

SEQ ID NO: 19 - DNA, 21 bp, Artificial Sequence
  Description of Artificial Sequence Synthetic oligonucleotide
tgtgcagggactgatgacggt

SEQ ID NO: 20 - DNA, 22 bp, Artificial Sequence
  Description of Artificial Sequence Synthetic oligonucleotide
ctgtgcatcgaagtttgcattt

SEQ ID NO: 21 - DNA, 22 bp, Artificial Sequence
  Description of Artificial Sequence Synthetic oligonucleotide
atgcacatctcaggatgacggt

SEQ ID NO: 22 - DNA, 22 bp, Artificial Sequence
  Description of Artificial Sequence Synthetic oligonucleotide
agttgcaggtcaggacccattt